(19) United States
(12) Patent Application Publication
(10) Pub. No.: US 2006/0058372 A1
(43) Pub. Date: Mar. 16, 2006

(54) N-Benzodioxolyl, n-benzodioxanyl and n-benzodioxepinyl arylcarboxamide derivatives and pharmaceutical compositions comprising them

(76) Inventors: Herve Dumas, Vaulx Milieu (FR); Jacques Barbanton, Brignais (FR); Francois Collonges, Beynost (FR); Jacques Decerprit, Neyron (FR); Jean-Yves Ortholand, Saint Jean De Niost (FR); David Benzies, Cornwall (GB); Stuart Cameron, Cornwall (GB); Richard J Foster, Cornwall (GB); Stefan M Guessregen, Devon (GB); Peter Kane, Cornwall (GB); Julia A H Lainton, Cornwall (GB); Avril A B Robertson, Cornwall (GB); Bernd Wendt, Weil (DE); Mark R Warne, Exeter (GB)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/532,587

(22) PCT Filed: Oct. 1, 2003
(86) PCT No.: PCT/EP03/10890

§ 371 Date: Apr. 25, 2005

(30) Foreign Application Priority Data
Oct. 25, 2002 (FR) 02/13419

(51) Int. Cl. A61K 31/335; C07D 321/10

(52) U.S. Cl. 514/450; 549/350

(57) ABSTRACT

The present invention relates to a compound of the formula (I): in which T, A, R, B Xi, Yi and n are as defined in Claim 1, and to the pharmaceutically usable derivatives, solvates and stereoisomers thereof, comprising a mixture thereof in all proportions, which can be used in the treatment of dyslipidaemia, and to pharmaceutical compositions comprising them.

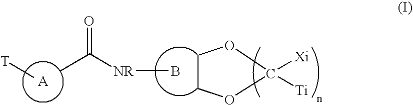

The invention relates to compounds that are inhibitors of microsomal triglyceride transfer protein (MTP), to pharmaceutical compositions comprising them and to their use in medicine.

Microsomal triglyceride transfer protein, known as MTP, is a transfer protein located in the reticulum of hepatocytes and enterocytes, which catalyses the assembly of biomolecules that transport triglycerides, the apo B lipoproteins.

The term apo B more particularly denotes apoprotein 48 of the intestine and apoprotein 100 of the liver.

Mutations in MTP or in the B apoproteins are reflected in man by very low levels or even an absence of apo B lipoproteins. The lipoproteins containing apo B (chylomicrons, very low density lipoproteins) and their metabolic residues (chylomicron remnants, low density lipoproteins) are recognised as being a major risk factor in the development of atherosclerosis, a major cause of death in industrialised countries. It is observed that, in individuals who are heterozygous for these mutations, levels reduced on average by a half are associated with a low cardiovascular risk (C. J. Glueck, P. S. Gartside, M. J. Mellies, P. M. Steiner, Trans. Assoc. Am. Physicians 90, 184 (1977)). This suggests that the modulation of the secretions of triglyceride-rich lipoproteins by means of MTP antagonists and/or of secretion of apo B might be useful in the treatment of atherosclerosis and more broadly of pathologies characterised by an increase in apo B lipoproteins.

Molecules that inhibit MTP and/or the secretion of apo B might thus be useful for the treatment of hypertriglyceridaemia, hypercholesterolaemia and diabetes-related dyslipidaemia, and also for the prevention of and treating obesity.

MTP inhibitors also functioning as apolipoprotein B (apo B) secretion inhibitors are known in the art.

Mention may be made of documents EP 887 345 and WO 98/23593, which describe compounds of the formula: 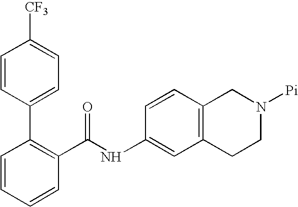
that have such properties.

Similarly, EP 1 099 701 describes compounds of the formula: 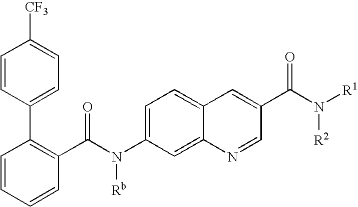
that can be used as apo B inhibitors.

Three other documents describe amides of the formula: 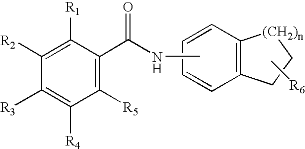
that are apo B and MTP inhibitors, these documents being: WO 01/53260, U.S. Pat. No. 6,197,798 and WO 00/05201

WO 97/26240 moreover describes compounds of the formula: 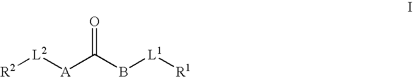
in which B is a group of fluorenyl or indenyl type. These compounds are MT inhibitors.

The invention provides compounds that are MTP inhibitors, which are also capable of inhibiting apolipoprotein B (apo B) secretion. None of the compounds described in the prior art contains the dioxacycloalkyl group of the compounds of the invention. The compounds of the invention are more specifically of the formula I: 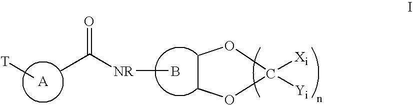
in which
A and B independently represent an optionally substituted phenyl nucleus; or an optionally substituted pyridyl nucleus;
T represents an optionally substituted, saturated and/or unsaturated aromatic carbocyclic nucleus; an optionally substituted, saturated and/or unsaturated aromatic heterocyclic nucleus; or
T represents a saturated and/or unsaturated aromatic carbocyclic nucleus which is fused to the nucleus A, is optionally substituted and is linked to two adjacent carbon atoms belonging to the nucleus A;
R represents a hydrogen atom; an optionally substituted saturated aliphatic hydrocarbon-based group; or an optionally substituted, saturated or unsaturated aromatic carbocyclic group;
n represents an integer chosen between 1, 2, 3, 4 and 5;
the radicals Xᵢ and Yᵢ are independently chosen from a hydrogen atom; a halogen atom; an optionally substituted, saturated and/or unsaturated aliphatic hydrocarbon-based group; an optionally substituted, saturated or unsaturated aromatic carbocyclic nucleus; a-u¹-COOL group, in which u¹ represents a bond or an alkylene group and L is an optionally substituted saturated aliphatic hydrocarbon-based group or an optionally substituted, saturated and/or unsaturated aromatic carbocyclic group; -u²-SiR¹R²R³, in which u² represents a bond, an alkylene group or an alkyleneoxy group in which the oxygen atom is linked to Si and R¹, R² and R³ independently represent an optionally substituted saturated aliphatic hydrocarbon-based group; -u³-OW, in which u³ represents a bond or an alkylene group and W may represent a hydrogen atom or is as defined above for L; u⁴-CO-G, in which u⁴ represents a bond, an alkylene group or an alkyleneoxy group in which the oxygen atom is linked to the carbonyl group and G is as defined above for L; -u⁵-CO—NH-J, in which u⁵ represents a bond, an alkylene group or an alkyleneoxy group in which the oxygen atom is linked to the carbonyl group and J is as defined above for L; or a radical Xi and a radical Yi both attached to the same carbon atom, together with this carbon atom, represent an optionally substituted saturated carbocyclic nucleus; and pharmaceutically usable derivatives, solvates and stereoisomers thereof comprising a mixture thereof in all proportions.

The carbocyclic and heterocyclic radicals include monocyclic and polycyclic radicals; these radicals preferably denote monocyclic, bicyclic or tricyclic radicals. In the case of polycyclic radicals, it should be understood that these radicals consist of monocycles fused in pairs (for example ortho-fused or peri-fused), i.e. having at least two carbon atoms in common. Preferably, each monocycle is 3- to 8-membered and better still 5- to 7-membered.

The cycloalkyl groups are an example of saturated carbocyclic radicals and preferably contain from 3 to 18 and better still from 3 to 10 carbon atoms, such as cyclopropyl, cyclobutyl, cyclopentyl, cyclohexyl, cycloheptyl, cyclooctyl, adamantyl or norbornyl radicals.

The aromatic carbocyclic groups are, for example, C₆-C₁₈ aryl groups and especially phenyl, naphthyl, anthryl and phenanthryl.

The heterocyclic groups contain heteroatoms, generally chosen from O, S and N, optionally in oxidised form (in the case of S and N).

Preferably, each of the monocycles constituting the heterocycle contains from 1 to 4 heteroatoms and better still from 1 to 3 heteroatoms. In a particularly preferred manner, each of the monocycles constituting the heterocycle is 5- to 7-membered.

The following are especially distinguished:
5- to 7-membered monocyclic heterocycles, for instance heteroaryls chosen from pyridine, furan, thiophene, pyrrole, pyrazole, imidazole, thiazole, isoxazole, isothiazole, furazane, pyridazine, pyrimidine, pyrazine, thiazines, oxazole, pyrazole, oxadiazole, triazole and thiadiazole, and also the saturated and unsaturated derivatives thereof. Examples of unsaturated 7-membered heterocycles are trithiatriazepines and trithiadiazepines. Examples of 5- to 7-membered saturated heterocycles are especially tetrahydrofuran, dioxolane, imidazolidine, pyrazolidine, piperidine, dioxane, morpholine, dithiane, thiomorpholine, piperazine, trithiane, oxepine and azepine;
bicyclic heterocycles in which each monocycle is 5- to 7-membered, for instance heteroaryls chosen from indolizine, indole, isoindole, benzofuran, benzothiophene, indazole, benzimidazole, benzothiazole, benzofurazane, benzothiofurazane, purine, quinoline, isoquinoline, cinnoline, phthalazine, quinazoline, quinoxaline, naphthyridines, pyrazolotriazine (such as pyrazolo-1,3,4-triazine), pyrazolopyrimidine and pteridine; and also the saturated and unsaturated derivatives thereof;
tricyclic heterocycles in which each monocycle is 5- to 7-membered, whether they are completely aromatic, for instance acridine, phenazine or carbazole, or not, such as saturated and unsaturated derivatives thereof, phenothiazine or phenoxazine.

The expression “saturated, unsaturated and/or aromatic carbocyclic radical” means that the same radical may contain a saturated carbocyclic portion and/or an unsaturated carbocyclic portion and/or an aromatic carbocyclic portion.

Similarly, the expression “saturated, unsaturated and/or aromatic heterocyclic radical” means that the same radical may contain a saturated heterocyclic portion and/or an unsaturated heterocyclic portion and/or an aromatic heterocyclic portion.

Examples of saturated and/or unsaturated aromatic carbocyclic nuclei include the following radicals:

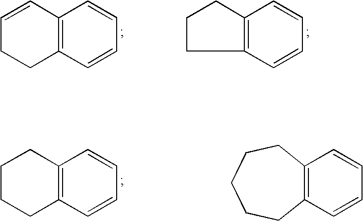

Examples of saturated, unsaturated and/or aromatic heterocyclic nuclei include the following: 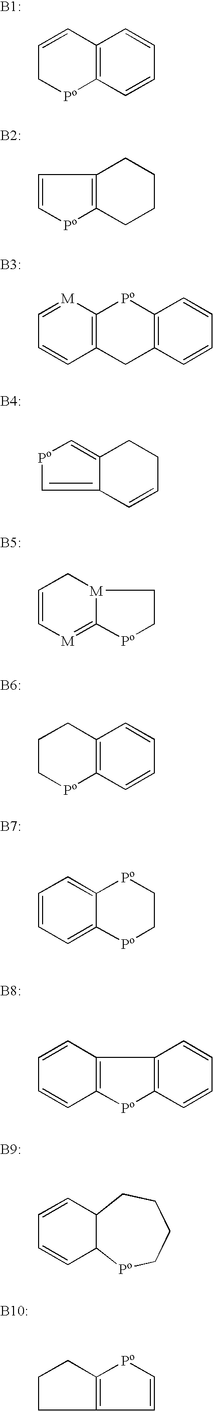 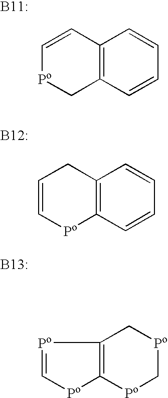
in which P^{O} represents O, S or SO₂ and M represents N or C. Preferably, in B1, P^{O} represents O; in B2, P^{O} represents O or S; in B3, P^{O} represents SO₂ or O and M represents C or N; in B4, P^{O} represents S; in B5, M represents N and P^{O} represents S; in B6, P^{O} represents O; in B7, P^{O} represents O; in B8, P^{O} represents O; in B9, P^{O} represents O; in B10, P^{O} represents S; in B11, P^{O} represents O; in B12, P^{O} represents O; in B13, P^{O} represents N.

If M or P^{O} represents N, this atom is preferably substituted by a hydrogen atom or by alkyl or alkylcarbonyl.

If T represents a saturated and/or unsaturated aromatic carbocyclic nucleus fused to the nucleus A, T and A are ortho-fused, the nucleus T being linked to two adjacent carbon atoms belonging to the nucleus A.

Thus, by way of example, T and A may together form one of the following radicals:

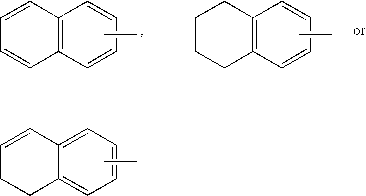

The term “aliphatic hydrocarbon-based group” means a linear or branched hydrocarbon-based chain, preferably of C₁-C₁₄ and better still C₁-C₁₀, for example C₁-C₆ or C₁-C₄.

If this chain is unsaturated, it contains one or more unsaturations, preferably one or two unsaturations. The unsaturations are of either ethylenic or acetylenic type. They are preferably ethylenic. The unsaturated chains contain at least two carbon atoms.

The unsaturated aliphatic hydrocarbon-based groups usually contain from 2 to 14 carbon atoms and better still from 2 to 10 carbon atoms, for example from 2 to 4 carbon atoms.

Examples of these groups include alkenyl groups and especially vinyl or allyl, and alkynyl groups, such as propargyl.

The alkyl groups are examples of saturated aliphatic hydrocarbon-based chains.

In the context of the invention, the term “alkyl” means a linear or branched hydrocarbon-based chain containing from 1 to 14 carbon atoms, preferably from 1 to 10 and better still from 1 to 6 carbon atoms, for example from 1 to 4 carbon atoms.

Examples of alkyl radicals are methyl, ethyl, propyl, isopropyl, butyl, isobutyl, t-butyl, pentyl, isopentyl, neopentyl, 2-methylbutyl, 1-ethylpropyl, hexyl, isohexyl, neohexyl, 1-methylpentyl, 3-methylpentyl, 1,1-dimethylbutyl, 1,3-dimethylbutyl, 2-ethylbutyl, 1-methyl-1-ethylpropyl, heptyl, 1-methylhexyl, 1-propylbutyl, 4,4-dimethylpentyl, octyl, 1-methylheptyl, 2-methylhexyl, 5,5-dimethylhexyl, nonyl, decyl, 1-methylnonyl, 3,7-dimethyloctyl and 7,7-dimethyloctyl.

The alkylene groups are divalent groups corresponding to the alkyl group above, but in which a hydrogen atom has been replaced by a bond.

The expression “optionally halogenated alkyl interrupted by one or more oxygen or sulfur atoms” means an alkyl chain in which one or more of the carbon-carbon, carbon-halogen or carbon-hydrogen bonds is interrupted by an oxygen or sulfur atom, it being understood that this chain does not contain two consecutive oxygen or sulfur atoms, or even an oxygen atom attached to a sulfur atom.

Examples of optionally halogenated alkyls interrupted by one or more oxygen or sulfur atoms are:
alkoxy;
thioalkoxy;
hydroxyalkyl;
alk^{o}-SH, in which alk^{o} is alkyl;
alk′-Calc-alk″, in which alk′ and alk″ are independently alkyl and Calc is O or S;
   or the corresponding radicals in which one or more of the alkyl or alkylene chains present are halogenated, for example perhalogenated.

Examples of the latter halogenated radicals are —OCF₃; OCF₂—CF₃; —CF₂—O—CF₃; —S—CF₃; —S—CF₂CF₃; or —CF₂—S—CF₃.

Haloalkyl radicals that may be mentioned include —CF₃; —CF₂—CF₃.

The term “halogen atom” means chlorine, bromine, iodine or fluorine.

According to one preferred embodiment of the invention, A and B independently represent an optionally substituted phenyl nucleus.

According to another preferred embodiment of the invention, B represents optionally substituted phenyl; and A represents optionally substituted pyridyl.

Preferred substituents of the nuclei A and B are halogen atoms, and alkyl and alkoxy radicals, in which the alkyl portion is as defined above, this alkyl portion preferably being C₁-C₆.

Preferably, T represents an optionally substituted monocyclic or bicyclic aryl nucleus, for example phenyl or naphthyl; or a monocyclic or bicyclic, saturated and/or unsaturated aromatic heterocyclic nucleus, containing 1 to 3 heteroatoms chosen from N, O and S, the said heterocyclic nucleus optionally being substituted; preferably, T represents a nucleus chosen from phenyl, pyrrolyl, phthalimidyl or succinimidyl, which is optionally substituted.

Preferred substituents are oxo, a halogen atom, alkyl which is optionally halogenated and/or optionally interrupted by one or more oxygen or sulfur atoms; -alk¹-O—CO—R⁴ in which alk¹ is an alkylene radical and R⁴ represents alkyl or alkylamino; -alk²-CO—O—R⁵ in which alk² is an alkylene radical and R⁵ is as defined above for R⁴; —CO—R⁶ in which R⁶ is as defined above for R⁴; hydroxyalkyl; -alk³-TT-Q in which alk³ represents alkylene, TT represents O or NH, and Q represents an optionally substituted arylalkyl nucleus; optionally substituted heteroarylalkyl; —CO—K in which K represents alkyl or alkoxy; or —SO₂—K in which K is as defined above; -alk⁴-O—CO—NH-alk⁵ in which -alk⁴ and alk⁵ independently represent alkylene; aminoalkyl; hydroxyalkyl, heteroarylalkyl, preferably imidazolylalkyl; and alkenyl.

Even more preferably, T represents phenyl, pyrrolyl, phthalimidyl or succinimidyl optionally substituted by one or more radicals chosen from:
alkyl optionally halogenated and/or optionally interrupted by one or more oxygen or sulfur atoms;
alk¹-O—CO—R⁴ in which alk¹ is an alkylene radical and R⁴ represents alkyl or alkylamino;
alk²-CO—O—R⁵ in which alk² is an alkylene radical and R⁵ is as defined above for R⁴;
CO—R⁶ in which R⁶ is as defined above for R⁴;
hydroxyalkyl;
heteroarylalkyl, preferably imidazolylalkyl; and
alkenyl.

Advantageously, R represents H or alkyl.

The compounds of the formula I in which n represents 1, 2 or 3 are also preferred.

Preferably, Xi and Yi are independently chosen from a hydrogen atom; a halogen atom; an alkyl group optionally interrupted by one or more oxygen or sulfur atoms; a hydroxyalkyl group; —COOL in which L is as defined above; -alk³-SiR¹R²R³ in which alk³ represents alkylene and R¹, R² and R³ are as defined above; -alk⁴-O—CO-alk⁵ in which alk⁴ and alk⁵ independently represent alkyl; -alk⁶-O—CO—NH-alk⁷ in which alk⁶ and alk⁷ independently represent alkyl.

One particular subgroup of compounds of the invention consists of the compounds for which A represents pyridyl; B represents phenyl; n represents 1, 2 or 3; R represents H; and Xi and Yi represent a hydrogen atom or a fluorine atom.

The radicals Xi, which are attached to different carbon atoms, are not all identical to each other.

Similarly, the radicals Yi, which are attached to different carbon atoms, are not all identical to each other.

One preferred subgroup of compounds of the invention consists of compounds for which the radicals Xi and Yi, attached to the same carbon atom, are identical and are both equal to a hydrogen atom or a fluorine atom.

Compounds that are particularly preferred are those in the examples.

The following compounds are more particularly preferred:
5-(4′-trifluoromethylbiphen-2-ylcarbonylamino)-2,2-difluorobenzo[1,3]-dioxole;
5-(4′-isopropylbiphen-2-ylcarbonylamino)-2,2-difluorobenzo[1,3]dioxole;
5-(4′-methoxybiphen-2-ylcarbonylamino)-2,2-difluorobenzo[1,3]dioxole;
5-(4′-trifluoromethoxybiphen-2-ylcarbonylamino)-2,2-difluorobenzo[1,3]-dioxole;
5-(4′-isopropylbiphen-2-ylcarbonylamino)benzo[1,3]dioxole;
5-(4′-ethyl-3-methylbiphen-2-ylcarbonylamino)-2,2-difluorobenzo[1,3]-dioxole;
5-(4′-ethylaminocarbonyloxyethylbiphen-2-ylcarbonylamino)-2,2-difluorobenzo[1,3]dioxole;
5-(4′-trifluoromethoxy-3-methylbiphen-2-ylcarbonylamino)-2,2-difluorobenzo[1,3]dioxole;
5-(4′-methoxycarbonylethylbiphen-2-ylcarbonylamino)-2,2-difluorobenzo-[1,3]dioxole;
4′-isopropylbiphenyl-2-carboxylic acid (3-methoxymethyl-2,3-dihydrobenzo[1,4]dioxin-6-yl)amide;
7-[(4′-isopropylbiphenyl-2-carbonyl)amino]-2,3-dihydrobenzo[1,4]dioxin-2-ylmethyl ethylcarbamate;
4′-ethylbiphenyl-2-carboxylic acid (2,2-difluorobenzo[1,3]dioxol-5-yl)-amide;
4′-trifluoromethoxybiphenyl-2-carboxylic acid benzo[1,3]dioxol-5-ylamide;
4′-(2-hydroxyethyl)biphenyl-2-carboxylic acid (2,2-difluorobenzo[1,3]-dioxol-5-yl)amide;
4′-isobutylbiphenyl-2-carboxylic acid (2,2-difluorobenzo[1,3]dioxol-5-yl)-amide;
4′-(2-methylpropenyl)biphenyl-2-carboxylic acid (2,2-difluorobenzo[1,3]-dioxol-5-yl)amide;
6-chloro-4′-isopropylbiphenyl-2-carboxylic acid (2,2-difluorobenzo[1,3]-dioxol-5-yl)amide;
6-chloro-4′-trifluoromethoxybiphenyl-2-carboxylic acid (2,2-difluorobenzo-[1,3]dioxol-5-yl)amide;
4′-(2-benzyloxyethyl)-6-methylbiphenyl-2-carboxylic acid (2,2-difluorobenzo[1,3]dioxol-5-yl)amide;
6-methoxy-4′-trifluoromethoxybiphenyl-2-carboxylic acid (2,2-difluorobenzo[1,3]dioxol-5-yl)amide;
6-methyl-4′-trifluoromethoxybiphenyl-2-carboxylic acid (2-methoxymethyl-2,3-dihydrobenzo[1,4]dioxin-6-yl)amide;
6-[(6-methyl-4′-trifluoromethoxybiphenyl-2-carbonyl)amino]-2,3-dihydrobenzo-[1,4]dioxin-2-ylmethyl ethylcarbamate;
2-[6′-(2,2-difluorobenzo[1,3]dioxol-5-ylcarbamoyl)-2′-methylbiphenyl-4-yl]-ethyl ethylcarbamate;
4′-ethylbiphenyl-2-carboxylic acid benzo[1,3]dioxol-5-ylamide.

The invention is directed not only towards the compounds of the formula I but also towards the salts thereof.

If the compound of the formula I contains an acid function, for example a carboxylic function, this acid can form a salt with a mineral or organic base.

As examples of salts with organic or mineral bases, mention may be made of the salts formed with metals and in particular alkali metals, alkaline-earth metals and transition metals (such as sodium, potassium, calcium, magnesium or aluminium) or with bases, such as ammonia or secondary or tertiary amines (such as diethylamine, triethylamine, piperidine, piperazine or morpholine) or with basic amino acids, or with osamines (such as meglumine) or with amino alcohols (such as 3-aminobutanol and 2-aminoethanol).

If the compound of the formula I contains a basic function, for example a nitrogen atom, this compound can form a salt with an organic or mineral acid.

The salts with organic or mineral acids are, for example, the hydrochloride, hydrobromide, sulfate, hydrogen sulfate, dihydrogen phosphate, citrate, maleate, fumarate, 2-naphthalenesulfonate and para-toluenesulfonate.

The invention also covers salts that allow a suitable separation or crystallisation of the compounds of the formula I, such as picric acid, oxalic acid or an optically active acid, for example tartaric acid, dibenzoyltartaric acid, mandelic acid or camphorsulfonic acid. However, a preferred subgroup of salts consists of salts of the compounds of the formula I with pharmaceutically acceptable acids or bases.

The invention also relates to the optically active forms (stereoisomers), enantiomers, racemic mixtures, diastereoisomers, hydrates and solvates of these compounds. The term “solvates of these compounds” means the addition of inert solvent molecules to the compounds, these solvates forming on account of their mutual force of attraction. Examples of solvates are monohydrates, dihydrates and alkoxides.

The term “pharmaceutically usable derivatives” means, for example, the salts of the compounds according to the invention and of prodrug compounds.

The term “prodrug derivatives” means compounds of the formula I that have been modified, for example with alkyl or acyl groups, sugars or oligopeptides, and that are rapidly cleaved in the body to form the active compounds according to the invention.

They also include biodegradable polymeric derivatives of the compounds according to the invention, as described, for example, in Int. J. Pharm. 115, 61-67 (1995).

The invention also relates to mixtures of the compounds of the formula I according to the invention, for example mixtures of two diastereoisomers, for example in proportions of 1:1, 1:2, 1:3, 1:4, 1:5, 1:10, 1:100 or 1:1000.

In a particularly preferred manner, they are mixtures of stereoisomeric compounds.

The compounds of the invention can be prepared by performing a process comprising the coupling of a carboxylic acid of the formula II: 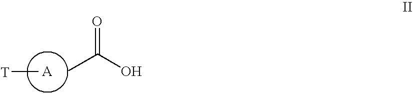
in which A and T are as defined above for formula I, optionally in activated form, with an amine of the formula III 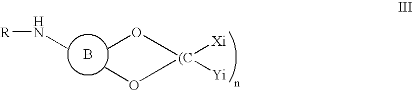
in which R, Xi, Yi, n and B are as defined above, in the presence of a base.

The term “coupling” means the formation of the corresponding amide bond.

To perform this coupling, inspiration may be taken from the reaction conditions described in the literature for peptide synthesis.

An activated derivative of the acid II is a compound in which the carboxylic function —COOH has been replaced by a more reactive function, such as —CO—K, in which K denotes a halogen atom (especially a chlorine atom), an imidazolide; p-nitrophenoxy; 1-benzotriazole; N—O-succinimide; acyloxy (such as pivaloyloxy); (C₁-C₄ alkoxy)carbonyloxy; dialkyl- or dicycloalkyl-O-ureide group.

If the compounds of the formula II are used in their free carboxylic acid form, the reaction is performed in the presence of a coupling agent, for instance a carbodiimide, optionally in the presence of an activating agent, for instance hydroxybenzotriazole or hydroxysuccinimide.

Representative coupling agents are dicycloalkyl- and dialkylcarbodiimides, carbodiimides that are soluble in an aqueous medium and especially dicyclohexylcarbodiimide, diisopropylcarbodiimide and (3-dimethylaminopropyl)-3-ethylcarbodiimide.

Use can be made more generally of any of the following coupling agents:
O-(7-azabenzotriazol-1-yl)-N,N,N′,N′-tetramethyluronium hexafluorophosphate (HBTU);
1-ethyl-3-(3′-dimethylaminopropyl)carbodiimide hydrochloride;
isobutyl chloroformate;
methanesulfonyl chloride;
bromotris(pyrrolidino)phosphonium hexafluorophosphate;
chloro-N,N,N′,N′-bis(tetramethylene)formamidinium tetrafluoroborate.

Examples of preferred inert solvents are especially optionally halogenated aliphatic and aromatic hydrocarbons (such as hexane, heptane, toluene, benzene, xylene, methylene chloride, chloroform, carbon tetrachloride, dichloroethane, chlorobenzene or dichlorobenzene); amides (such as formamide, N,N-dimethylformamide, dimethylacetamide, N-methyl-2-pyrrolidinone and hexamethylphosphorylamide); and nitriles (such as acetonitrile or isobutyronitrile).

The reaction is advantageously performed in the presence of a base chosen from pyridine, 4-dimethylaminopyridine (4-DMAP), 2,6-di-tert-butylpyridine, 1,8-diazabicyclo[5.4.0]undec-7-ene (DBU), 1,5-diazabicyclo[4.3.0]non-5-ene (DBN), 1,4-diazabicyclo[2.2.2]octane (DABCO or triethylenediamine), triethylamine, N,N-diisopropylethylamine, and Hünig's base or N-methylmorpholine.

If the carboxylic acid is used without preactivation of the carboxylic function, the two reagents II and III are preferably reacted together in equimolar amounts.

If an activated form of the carboxylic acid is used, equimolar amounts of the acid II and of the amine III are, in this case also, preferably used.

However, it is possible to use the acid or its activated form in slight excess relative to the amount of amine present: by way of example, the molar ratio of the carboxylic acid or of its activated form to the amine ranges between 1 and 3 and preferably between 1 and 2, for example between 1 and 1.5.

The reaction temperature is advantageously maintained between room temperature (15 and 35° C.) and the reflux point of the solvent. The reaction temperature is between 15 and 40° C. and better still between 20 and 30° C.

According to one preferred embodiment of the invention, the activated form of the carboxylic acid II that it is used is a chloride of this acid.

The chloride of the carboxylic acid II is prepared by the action of oxalyl chloride on the carboxylic acid II.

This reaction is advantageously performed at low temperature, for example between −20 and 15° C., preferably between −5° C. and 10° C. and better still between 0 and 5° C., in a polar aprotic solvent, such as an optionally halogenated aliphatic or aromatic hydrocarbon as defined above (for example dichloromethane); an amide as defined above, preferably N,N-dimethylformamide; a nitrile as defined above, preferably acetonitrile.

Advantageously, an excess of oxalyl chloride is reacted with the carboxylic acid II. The acid chloride of the carboxylic acid II can be prepared in any other conventional manner, such as by the action of SOCl₂, PCl₃ or PCl₅.

The amines of the formula III are readily prepared by a person skilled in the art by carrying out methods known to those skilled in the art.

By way of illustration, scheme 1 below retraces the steps for the preparation of an amino alcohol of the formula III, in which R represents H, n represents 2, a first —CXY represents —CH₂ and a second —CXY— represents —CH(CH₂—OSi R¹R²R³)—.

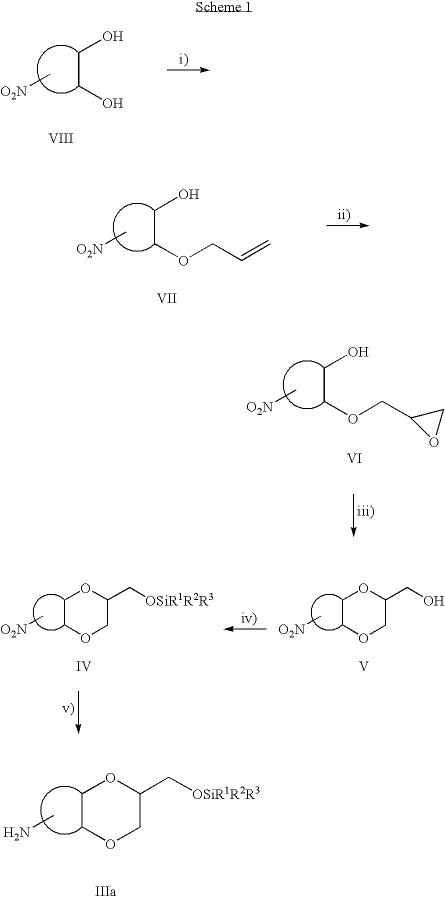

In scheme 1, R¹, R² and R³ are as defined above.

In step i), a compound of the formula VIII is reacted, in the presence of a base, with an allylic derivative of the formula IX:
CH₂═CH—CH₂-Lv
in which Lv is a leaving group, such as a halogen atom, preferably a bromine atom; arylsulfonyl optionally substituted by alkyl (such as toluenesulfonyl); or optionally halogenated alkylsulfonyl (such as mesyl or CF₃—SO₂—).

This reaction can be performed in any polar solvent, such as an optionally halogenated aliphatic or aromatic hydrocarbon, an amide or a nitrile, such as those defined above; or in an ether (such as diethyl ether, diisopropyl ether, tetrahydrofuran, dioxane, dimethoxyethane or diethylene glycol dimethyl ether); or a ketone (such as acetone, methyl ethyl ketone, methyl isobutyl ketone, isophorone or cyclohexanone).

According to one preferred embodiment of the invention, the base is a mineral base, such as NaOH, KOH, NaHCO₃, Na₂CO₃, KHCO₃ or K₂CO₃.

The reaction temperature is preferably between 15 and 40° C. and better still between 20 and 30° C.

A slight excess of reagent IX is usually used relative to the amount of a compound VIII used.

Thus, the molar ratio of compound IX to compound VIII preferably ranges between 1 and 3 and better still between 1 and 2. Similarly, the molar ratio of the base to compound VIII ranges between 1 and 3 and better still between 1 and 2.

In step ii), oxidation of the terminal double bond of compound VII is performed. To do this, an oxidising agent, such as meta-chloroperbenzoic acid can be used.

The reaction is preferably performed in a polar aprotic solvent, such as one of those defined above. The solvent is preferably a halogenated aliphatic hydrocarbon, such as dichloromethane.

This reaction is advantageously performed at room temperature, i.e. between 15 and 35° C.

If meta-chloroperbenzoic acid is used as oxidising agent, this agent is used in slight excess relative to the amount of compound VII. In this case also, a molar ratio of the oxidising agent to compound VII ranges between 1 and 3, for example between 1 and 2.

In step iii), the epoxide of the formula VI is treated by the action of a base, such as an alkali metal hydride or an alkali metal alkoxide. Preferred examples of alkali metal alkoxides included sodium or potassium methoxide, ethoxide or tert-butoxide. The base is more preferably sodium methoxide.

If the base is an alkali metal alkoxide, the reaction is preferably performed in the corresponding alkanol.

The temperature depends more particularly on the base chosen.

If the base is an alkali metal alkoxide, the process will be performed, for example, at room temperature, i.e. between 15 and 35° C.

A large excess of base can usually be used relative to the amount of epoxide present, for example from 3 to 10 equivalents and preferably from 4 to 6 equivalents.

In step iv), the silyl derivative IV is prepared in a manner known per se. The corresponding compound of the formula X
Lv-SiR¹R²R³ X
in which Lv, R₁, R₂ and R₃ are as defined above, is generally reacted with a compound of the formula V, in the presence of a base, such as an organic base.

Examples of suitable organic bases are N-methylmorpholine, triethylamine, tributylamine, diisopropylethylamine, dicyclohexylamine, N-methylpiperidine, pyridine, 4-(1-pyrrolidinyl)pyridine, picoline, 4-(N,N-dimethylamino)pyridine, 2,6-di-t-butyl-4-methylpyridine, quinoline, N,N-dimethylaniline and diethylaniline. Preferably, triethylamine is used as a mixture with 4-(N,N-dimethylamino)pyridine.

If Lv represents a halogen atom, and more particularly a chlorine atom, the reaction is performed in a polar aprotic solvent, for instance a halogenated aliphatic hydrocarbon, such as dichloromethane.

This reaction is advantageously performed at room temperature, for example between 15 and 35° C.

An excess of compound X relative to the amount of compound V is conventionally used. The molar ratio of the amount of silyl derivative X to compound V preferably ranges between 1 and 2 equivalents, for example between 1 and 1.5 equivalents.

In step v), hydrogenation of the nitro function to an amino function is performed. This reaction is performed, for example, under catalytic conditions, at a temperature of between 15 and 35° C.

The catalyst can be, for example, palladium-on-charcoal, and the solvent a C₁-C₄ alkanol, such as ethanol or methanol.

The compound obtained of the formula IIIa is a compound of the formula III from which can be prepared many other compounds of the formula III, via simple chemical conversion.

As a variant, compounds of the formula III can be prepared by converting the compound of the formula V and then by hydrogenating the nitro function to an amino function.

By way of illustration, scheme 2 shows the preparation of an alkoxylated derivative of the formula IIIb: 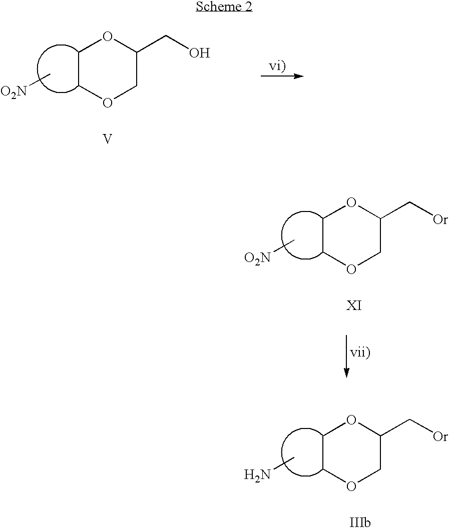
in which r represents C₁-C₁₄ alkyl.

In step vi), the alkylation of compound V is performed. This alkylation reaction can be performed under standard conditions, for example by the action of an alkyl iodide or more generally an alkyl halide in the presence of an alkali metal hydride, in a strongly polar aprotic solvent.

In a particularly preferred manner, the base is sodium hydride. Other hydrides that can be used are, for example, such as those defined above.

A temperature of between 10 and 30° C. and preferably between 20 and 25° C. is particularly suitable for this reaction.

If the reagent is an alkyl iodide and the base is a sodium hydride, the solvent is preferably dimethylformamide.

It is desirable for the base and the alkyl iodide to be present in excess in the reaction medium. Thus, for example, the sodium hydride is present in a proportion of from 1.5 to 3 molar equivalents relative to compound V, and the alkyl iodide is present in a proportion of from 3 to 10 molar equivalents relative to compound V.

The hydrogenation reaction in step vii can advantageously be performed under the same conditions as described above for step v).

The compounds of the formula IIIa and the derivatives thereof of the formula IIIc below form an integral part of the invention:

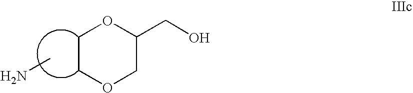

The derivatives IIIc can be obtained (i) either by catalytic hydrogenation under conditions similar to those described above, (ii) or by deprotection the of the hydroxyl function of compound IIIa, for example by the action of tetrabutylammonium fluoride under the conditions described in the literature, for example at room temperature (15-35° C.), in a solvent of ether type, such as tetrahydrofuran, by the action of a large excess of tetrabutylammonium fluoride (2 to 10 equivalents).

If A and T independently represent an optionally substituted phenyl group, the compounds of the formula II can be prepared by carrying out reaction scheme 3 below: 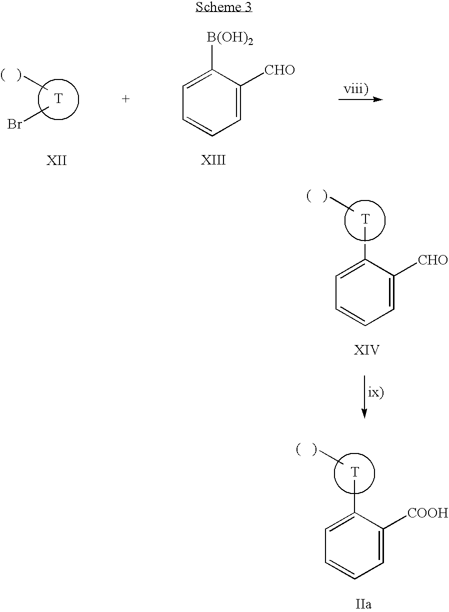
in which T represents phenyl; and
( [symbol] denotes the possible substituent(s) of T.

In step viii), the coupling of compounds XII and XIII can be performed in the presence of caesium fluoride and Pd(PPh₃)₄ or an equivalent palladium 0 complex.

The reaction is preferably performed at a temperature of between −10° C. and 10° C. and better still between −5° C. and 5° C.

As solvent, it is desirable to perform the process in a polar aprotic solvent, such as an ether and more particularly dimethyl ether or any of the ethers mentioned above.

A slight excess of the bromo derivative is recommended to perform this reaction.

Thus, it is desirable for the molar ratio of the bromo derivative XII to the aldehyde XIII to range between 1 and 3, preferably between 1 and 2 and better still between 1 and 1.5.

The palladium complex is present in the reaction medium in catalytic amount. A molecular ratio of the palladium complex to compound XIII of less than 0.1 and preferably less than 0.7 is particularly suitable.

As regards the molar ratio of the CsF to compound XIII, it ranges between 1 and 5, preferably between 2 and 4 and better still between 2 and 3.

Compound XIV is oxidised to compound IIa by the action of an oxidising agent.

Oxidising agents that may be chosen include any oxidising agent known in the art for oxidising an aldehyde function to a carboxylic acid function.

A particularly preferred oxidising agent that may be mentioned is Jones' reagent (CrO₃/H₂SO₄).

The solvent that can be used for this reaction is preferably a water-miscible polar solvent, the Jones' reagent being an aqueous 98% solution of CrO₃ in H₂SO₄. The solvent is preferably acetone.

According to one preferred embodiment, the reaction temperature is maintained between −10° C. and +10° C. and preferably between −5° C. and +5° C.

The amount of oxidising agent ranges between 1 and 10 and better still between 2 and 5 molar equivalents relative to the amount of aldehyde used.

As a variant, the oxidation reaction of compound XIV to compound IIa can be performed by the action of potassium permanganate.

The molar ratio of potassium permanganate to compound XIV advantageously ranges between 1 and 5, preferably between 1 and 3 and better still between 1.3 and 1.8.

The reaction is performed, for example, in one-phase aqueous medium, such as a mixture of water and acetone in a proportion ranging between 20/80 and 80/20.

The reaction temperature is generally between 10 and 50° C. and better still between 20 and 40° C., for example between 30 and 35° C.

If A and T independently represent an optionally substituted phenyl group, the corresponding compounds of the formula II can be prepared by carrying out reaction scheme 4 below: 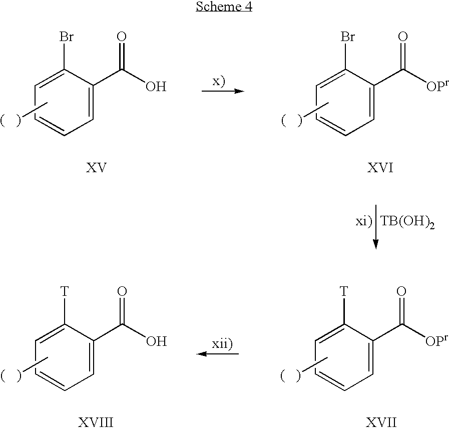
in which T represents optionally substituted phenyl and the phenyl group that represents A may be optionally substituted, where ( )- denotes the possible substituent(s) in A.

In step x), the carboxylic function of compound XV is protected by a temporary protecting group P^{r}.

Such groups are described especially in “Protective Groups in Organic Synthesis”. Greene T. W. and Wuts P. G. M, published by John Wiley and Sons, 1991, and in “Protecting Groups” Kocienski P. J., 1994, Georg Thieme Verlag.

More preferably, the group pr is an alkyl group and the carboxylic function is protected in the form of an ester.

The esterification reaction can be performed by simple reaction of the carboxylic acid XV with the corresponding alcohol P^{r}—OH in which pr represents alkyl, such as C₁-C₄ alkyl and better still methyl, and this reaction takes place in the presence of an acid catalyst, such as a sulfonic acid.

Such acids are especially optionally halogenated alkylsulfonic acids (for example methylsulfonic acid and trifluoromethylsulfonic acid), and arylsulfonic acids optionally substituted by alkyl on the aryl nucleus (for example paratoluenesulfonic acid).

In strict terms, a stoichiometric ratio of the acid catalyst to the alcohol is sufficient.

The solvent is generally the alcohol used as reagent, which is then present in large excess.

The reaction temperature under the abovementioned conditions is usually maintained between 40° C. and 150° C.; this temperature is advantageously the reflux point of the solvent.

In step xi), coupling is performed between compound XVI and TB(OH)₂, which is performed in the presence of a palladium 0 complex, such as Pd(PPh₃)₄, and a base, preferably a mineral base, such as an alkali metal hydroxide (for example sodium or potassium hydroxide), an alkali metal bicarbonate (sodium or potassium bicarbonate) or an alkali metal or alkaline-earth metal carbonate (for example sodium or potassium carbonate).

Suitable solvents are polar aprotic solvents, such as those mentioned above. Among these, nitrites and especially acetonitrile are especially preferred.

This reaction is generally performed by setting the molar ratio of TB(OH)₂ to the compound of the formula XVI between 1 and 3, preferably between 1 and 2 and better still between 1 and 1.4.

Similarly, the base is used in an amount such that the molar ratio of the base to the compound of the formula XVI ranges between 1 and 3, for example between 1 and 2 and better still between 1 and 1.5.

The palladium (0) complex used is present in the reaction medium in catalytic amount.

Thus, the molar ratio of the said complex to the compound of the formula XVI preferably ranges between 0.01 and 0.1.

In step xii), the ester of the formula XVII is deprotected. The reaction conditions will be readily established by a person skilled in the art as a function of the protecting group of the carboxylic function. With this aim, a person skilled in the art may refer to the publications mentioned above, namely Protective Groups in Organic Synthesis and Protecting Groups by Kocienski.

If the group Pr is an alkoxy group, the carboxylic function is readily freed by the action of a base, preferably one of the mineral bases mentioned above.

By way of illustration, the use of sodium hydroxide at a temperature of between 30 and 100° C. in an aqueous alcoholic medium (such as a mixture of a C₁-C₄ lower alcohol-methanol-in water) is suitable.

The compounds of the formula I bearing particular functions Xᵢ and/or Yᵢ can be obtained by simple conversion of the corresponding compounds of the formula I bearing suitable precursor functions.

By way of illustration, a compound of the formula I in which Xi and/or Yi represent u⁴-CO-G, in which u⁴ is alkyleneoxy and G represents a saturated hydrocarbon-based aliphatic group can be prepared from the corresponding compound of the formula I in which Xi and/or Yi represents u³-OW, in which u³ is alkylene and W represents H, by acylation under standard conditions.

Thus, the —CH₂—OH group can be readily converted into a —CH₂—O—CO—CH₃ group by the action of Ac₂O in the presence of a base, for example by the action of Ac₂O in pyridine.

By way of additional example, it is possible to convert a function -u³-OW in which u³ is alkylene and W represents H into a function -u⁵-CO—NH-J in which u⁵ is alkyleneoxy and in which J is an alkyl group.

To do this, the appropriate alkylisonitrile is reacted with the corresponding compound of the formula I containing at least one function Xi and/or Yi=u³-OW.

By way of example, the —CH₂—OH function is converted into a CH₂—O—CO-NEt function by the action of EtNCO in the presence of diisopropylethylamine in dichloromethane at 40° C.

Another example is that of the conversion of the function u³-OW in which u³ is alkylene and W represents H into a function u³-OW in which u³ is as defined above and W is alkyl. This conversion can be performed by the action of a basic hydride, such as sodium hydride on an alkyl halide (methyl iodide) in a solvent, such as dimethyl sulfoxide. This reaction can bring about the simultaneous methylation of any amino function present in the compound of the formula I.

The compounds of the formula Ia in which T represents optionally substituted phenyl; A represents optionally substituted phenyl; and ( )- denotes the possible substituent(s) in A, can be prepared by coupling a bromide of the formula XXI: 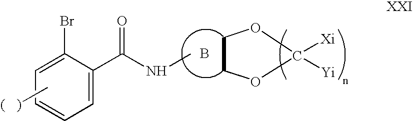
in which ( )-, B, Xi, Yi and n are as defined above, with a compound of the formula TB(OH)₂ in which T is as defined above, in the presence of a base and a palladium 0 complex, so as to synthesise the expected compound of the formula Ia:

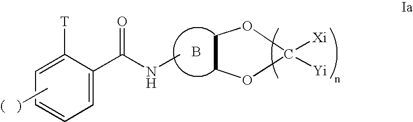

As bases that can be used, mineral bases, such as NaOH, KOH, potassium carbonate, sodium carbonate, potassium hydrogen carbonate or sodium hydrogen carbonate are preferred.

Preferably, the palladium complex is tetrakis(triphenylphosphine)palladium (0).

This reaction is preferably performed in a polar aprotic solvent, such as a nitrile, for example acetonitrile. The reaction medium is refluxed at a temperature of between 50 and 120° C. and preferably between 75 and 90° C.

Preferably, stoichiometric amounts of the reagents will be used in the presence of TB(OH)₂ and of the compound of the formula XXI, TB(OH)₂ possibly being used in excess. Usually, the molar ratio of TB(OH)₂ to the compound of the formula XXI ranges between 1 and 2 equivalents. Similarly, the base will be used in a proportion of from 1 to 2 equivalents relative to the compound of the formula XXI.

Finally, a catalytic amount of the palladium 0 complex is generally sufficient. This catalyst will be present, for example, in the reaction medium, in a proportion of from 1 to 5 mol % relative to the compound of the formula XXI.

The intermediate compound of the formula XXI can be prepared by reacting the chloride of the formula XIX with the amine of the formula XX according to the following reaction scheme: 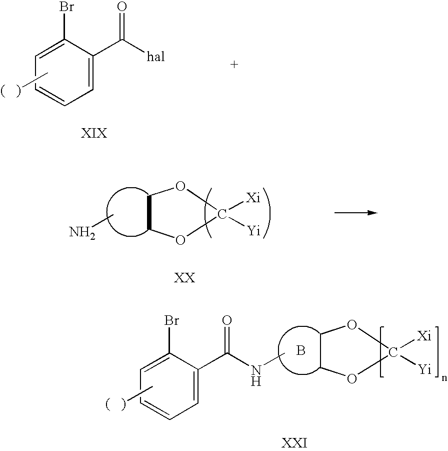
in which formulae ( )-, Xi, Yi and n are as defined above and hal represents a halogen atom,
this reaction preferably being performed in the presence of a base.

Examples of bases that can be used are especially organic bases, such as triethylamine, pyridine, 4-dimethylaminopyridine, 2,6-di-tert-butylpyridine, 1,8-diazabicyclo[5.4.0]undec-7-ene (DBU), 1,5-diazabicyclo[4.3.0]non-5-ene (DBN), 1,4-diazabicyclo[2.2.2.]octane (DABCO or triethylenediamine) or any mixture thereof. In a more particularly preferred manner, 4-dimethylaminopyridine will be used.

The reaction is preferably performed in a solvent, for instance a nitrile, such as acetonitrile.

The addition of compound XIX to compound XX is performed at low temperature, preferably at a temperature of between −10 and +10° C., for example between −5 and +5° C. The reaction medium is then maintained for the required time at room temperature (i.e. at a temperature of between 15 and 30° C. and especially between 18 and 25° C.).

The molar ratio of compound XIX to compound XX is preferably between 1 and 1.5 equivalents and better still between 1 and 1.3 equivalents.

The base will be introduced into the reaction medium in a proportion of from 1 to 3 equivalents relative to compound XX and better still in a proportion of from 1.3 to 2 equivalents. If the base is a mixture of triethylamine and 4-dimethylaminopyridine, the said base is preferably used in catalytic amounts.

The compound of the formula XIX can be obtained in a conventional manner from the corresponding carboxylic acid of the formula XV, for example by the action of oxalyl chloride in a polar aprotic solvent and preferably in a halogenated aliphatic hydrocarbon, such as dichloromethane, chloroform or carbon tetrachloride. For this reaction, the temperature of the reaction medium is preferably maintained between −10° C. and +10° C. and especially between −5° C. and +5° C., and the temperature is then adjusted to between 30 and 80° C. and better still between 40 and 60° C.

The compounds of the formula TB(OH)₂ in which T represents optionally substituted aryl can be simply prepared by carrying out reaction scheme 5 below, 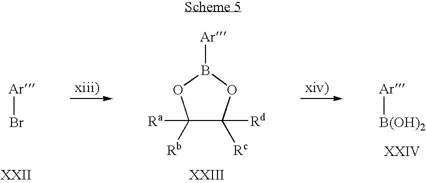
in which formulae Ar′″ represents aryl; R^{a}, R^{b}, R^{c} and R^{d} independently represent C₁-C₆ alkyl.

In step xiii), compound XXII is reacted with a borane of the formula XXV: 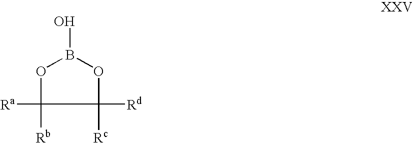
in which R^{a}, R^{b}, R^{c} and R^{d} are as defined above, in the presence of a base, such as an organic base of the type mentioned above and preferably in the presence of triethylamine, and in the presence of a palladium II complex, for instance a palladium II chloride, such as bis(triphenylphosphine)palladium (II) chloride.

The molar ratio of compound XXV to compound XXII preferably ranges between 1 and 2 equivalents, for example between 1.2 and 1.8 equivalents. The palladium II chloride is present in catalytic amount in the reaction medium, for example in a proportion of from 2 mol % to 5 mol % relative to the compound of the formula XXII.

As reaction solvent, it is desirable to select a linear or cyclic ether, such as diethyl ether, di-tert-butyl ether, dioxane or tetrahydrofuran, preferably dioxane. The reaction is preferably performed at room temperature, and the reaction medium is then brought to a higher temperature, for example between 50 and 150° C. and better still between 80 and 120° C.

In step (xiv), the expected compound of the formula XXIV is obtained by the action of sodium periodate in the presence of ammonium acetate in aqueous medium on the compound of the formula XXIII. The reaction medium that will be selected, for example, is a mixture of a ketone, such as acetone and water or a mixture of a lower (C₁-C₄) alcohol and water.

A suitable temperature is room temperature (15 to 35° C.), such as a temperature of between 20 and 25° C. Advantageously, the sodium periodate is used in a proportion of from 2 to 5 equivalents and better still in a proportion of 3 to 4 equivalents relative to the starting compound XXIII. The molar ratio of the sodium periodate to the ammonium acetate is usually 1. More generally, the amount of ammonium acetate will be set at between 2 and 5 equivalents and better still between 3 and 4 equivalents relative to compound XXIII.

Some of the intermediate compounds of the formulae XXI, XIV, IIa, IIIb, XI, IIIa and IV are novel and constitute another aspect of the invention.

More specifically, the invention relates to one of the following subgroups of intermediate compounds:
the compounds of the formula XXIa: 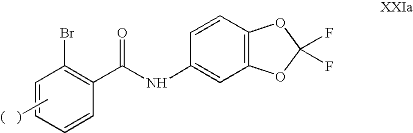
   in which ( [symbol] denotes the possible substituent(s) on the phenyl group to which ( [symbol] is attached, which are chosen from halogen, alkyl and alkoxy, and especially those for which ( [symbol] represents methyl;
the compounds of the formula XIVa: 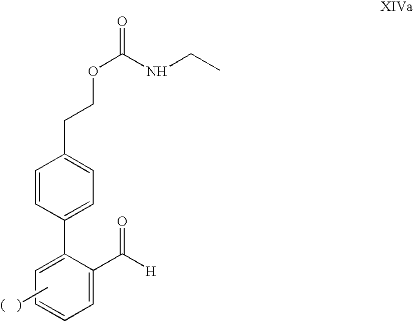
   in which ( [symbol] denotes the possible substituent(s) on the phenyl group to which ( [symbol] is attached, which are chosen from halogen, alkyl and alkoxy, and especially those for which ( [symbol] denotes a hydrogen atom or a methyl group;
the compounds of the formula II b: 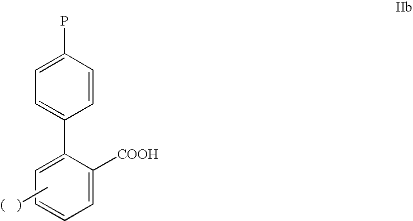
   in which
P is chosen from —OCF₃ provided that ( [symbol] does not represent hydrogen; —CO—CH(CH₃)₂; —(CH₂)₂—O—CO—CH₃; —(CH₂)₂—CO—O—CH₃; and —(CH₂)₂—O—CO—NH—CH₂—CH₃;
( [symbol] denotes the possible substituent(s) on the phenyl group to which ( [symbol] is attached, which are chosen from hydrogen, halogen, such as chlorine, alkyl, such as methyl, and alkoxy, such as methoxy, and especially chosen from:
6-methyl-4′-trifluoromethoxybiphenyl-2-carboxylic acid;
6-methoxy-4′-trifluoromethoxybiphenyl-2-carboxylic acid;
6-chloro-4′-trifluoromethoxybiphenyl-2-carboxylic acid;
4′-isobutyrylbiphenyl-2-carboxylic acid;
4′-(2-acetoxyethyl)biphenyl-2-carboxylic acid;
4′-(2-methoxycarbonylethyl)biphenyl-2-carboxylic acid;
4′-(2-ethylcarbamoyloxyethyl)biphenyl-2-carboxylic acid;
4′-(2-ethylcarbamoyloxyethyl)-6-methylbiphenyl-2-carboxylic acid;
   the compounds of the formula IIId: 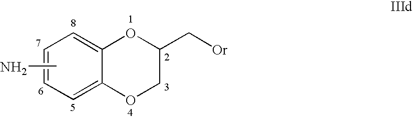
      in which r represents (C₁-C₆)alkyl, preferably methyl, and NH₂ is located in position 6 or 7, with the exclusion of 2-ethoxymethyl-2,3-dihydro-benzo[1,4]dioxin-7-ylamine,
      and especially those chosen from:
3-methoxymethyl-2,3-dihydrobenzo[1,4]dioxin-6-ylamine; and
2-methoxymethyl-2,3-dihydrobenzo[1,4]dioxin-6-ylamine;
   the compounds of the formula XI a: 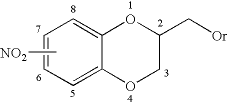
      in which r represents (C₁-C₆)alkyl, preferably methyl, and NO₂ is located in position 6 or 7, with the exclusion of 2-ethoxymethyl-7-nitro-2,3-dihydro-benzo[1,4]-dioxine,
      and especially those chosen from:
2-methoxymethyl-7-nitro-2,3-dihydrobenzo[1,4]dioxine,
2-methoxymethyl-6-nitro-2,3-dihydrobenzo[1,4]dioxine;
   the compounds of the formula IIIe: 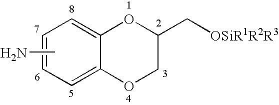
      in which R¹, R² and R³ independently represent (C₁-C₆)alkyl and —NH₂ is located in position 6 or 7,
      and especially those chosen from:
3-(tert-butyldimethylsilanyloxymethyl)-2,3-dihydrobenzo[1,4]dioxin-6-yl-amine, and
2-(tert-butyldimethylsilanyloxymethyl)-2,3-dihydrobenzo[1,4]dioxin-6-yl-amine;
   the compounds of the formula IVa: 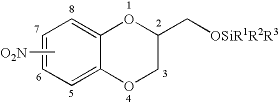
      in which R¹, R² and R³ independently represent (C₁-C₆)alkyl; and NO₂ is located in position 6 or 7,
      and especially those chosen from:
tert-butyldimethyl(7-nitro-2,3-dihydrobenzo[1,4]dioxin-2-ylmethoxy)-silane;
tert-butyldimethyl(6-nitro-2,3-dihydrobenzo[1,4]dioxin-2-ylmethoxy)silane.

According to another of its aspects, the invention relates to pharmaceutical compositions comprising one or more compounds of the formula I according to the invention, in combination with one or more excipients.

These compositions can be administered orally in the form of immediate-release or controlled-release tablets, gel capsules or granules, intravenously in the form of an injectable solution, transdermally in the form of an adhesive transdermal device, or locally in the form of a solution, cream or gel.

A solid composition for oral administration is prepared by adding to the active principle a filler and, where appropriate, a binder, a disintegrating agent, a lubricant, a colorant or a flavour enhancer, and by forming the mixture into a tablet, a coated tablet, a granule, a powder or a capsule.

Examples of fillers include lactose, corn starch, sucrose, glucose, sorbitol, crystalline cellulose and silicon dioxide, and examples of binders include poly(vinyl alcohol), poly(vinyl ether), ethylcellulose, methylcellulose, acacia, gum tragacanth, gelatin, Shellac, hydroxypropylcellulose, hydroxypropylmethylcellulose, calcium citrate, dextrin and pectin. Examples of lubricants include magnesium stearate, talc, polyethylene glycol, silica and hardened plant oils. The colorant can be any colorant permitted for use in medicaments. Examples of flavour enhancers include cocoa powder, mint in herb form, aromatic powder, mint in oil form, borneol and cinnamon powder. Needless to say, the tablet or granule can be suitably coated with sugar, gelatine or the like.

An injectable form comprising the compound of the present invention as active principle is prepared, where appropriate, by mixing the said compound with a pH regulator, a buffer agent, a suspension agent, a solubiliser, a stabiliser, a tonicity agent and/or a preserving agent, and by converting the mixture into a form for intravenous, subcutaneous or intramuscular injection, according to a conventional process. Where appropriate, the injectable form obtained can be lyophilised via a conventional process.

Examples of suspension agents include methylcellulose, polysorbate 80, hydroxyethylcellulose, acacia, powdered gum tragacanth, sodium carboxymethylcellulose and polyethoxylated sorbitan monolaurate.

Examples of solubilisers include castor oil solidified with polyoxyethylene, polysorbate 80, nicotinamide, polyethoxylated sorbitan monolaurate and the ethyl ester of castor oil fatty acid.

In addition, the stabiliser includes sodium sulfite, sodium metasulfite and ether, while the preserving agent includes methyl p-hydroxybenzoate, ethyl p-hydroxybenzoate, sorbic acid, phenol, cresol and chlorocresol.

The compounds of the formula I and the pharmaceutical compositions of the invention are useful as microsomal triglyceride transfer protein (MTP) inhibitors. As such, they can be used in the treatment of hypercholesterolaemia, hypertriglyceridaemia, hyperlipidaemia, pancreatitis, hyperglycaemia, obesity, atherosclerosis and diabetes-related dyslipidaemia.

Thus, according to yet another of its aspects, the invention relates to the use of a compound or a pharmaceutical composition according to the invention for the preparation of a medicament that inhibits microsomal triglyceride transfer protein.

The compounds of the invention also allow inhibition of the secretion of the B apoproteins (apo B).

The following tests were performed to demonstrate the inhibition of MTP activity and the inhibition of apo B secretion.

Analysis of the Inhibition of MTP Activity

The inhibition of the activity of microsomal triglyceride transfer protein (MTP) was tested by using the following operating protocol.

The inhibition of MTP activity with a compound can be quantified by observing the inhibition of the transfer of a labelled triglyceride, from a donor particle to an acceptor particle, in the presence of MTP. The procedure for the preparation of MTP is based on the method by Wetterau and Zilversmit (Biochem. Biophys. Acta (1986) 875: 610). A few grams of golden hamster liver are taken and then rinsed several times in a 250 mM sucrose solution at 0° C. All the following steps proceed at +4° C. A homogenate at a concentration of 50% in 250 mM sucrose is prepared using a Teflon mill and then centrifuged for 10 minutes at 10 000×g at +4° C. The supernatant is then centrifuged at 105 000×g for 75 minutes at +4° C. The supernatant is discarded and the microsomal pellet is taken up in 3 ml (per g of starting liver) of Tris/HCl 150 mM pH 8.0. 1 ml aliquot fractions are stored at −80° C. until the time of use.

After thawing a fraction of microsomes (1 ml), 12 ml of refrigerated Tris/HCl 50 mM, KCl 50 mM, MgCl₂ 5 mM pH 7.4 buffers and 1.2 ml of deoxycholate (0.54% in water) are added. After incubation for 30 minutes at +4° C. with gentle agitation, the suspension is centrifuged at 105 000×g for 75 minutes. The supernatant comprising the soluble MTP is dialysed against Tris/HCl 150 mM, NaCl 40 mM, EDTA 1 mM, 0.02% sodium azide pH 7.4 buffer (5 times one litre over 2-3 days). The MTP is stored at +4° C., is stable for at least 30 days and is used in unmodified form in the test.

The donor particles (liposomes) are prepared from 208 μl of L-phosphatidylcholine at a concentration of 10 mg/ml in chloroform, and 480 μl of [3H]-triolein at a concentration of 0.5 mCi/ml in toluene. After stirring, the solution is evaporated under nitrogen, taken up in 6 ml of Tris/HCl 50 mM, KCl 50 mM, MgCl₂ 5 mM pH 7.4 buffer and incubated in an ultrasound bath for 30 minutes at room temperature. The liposomes are stored at +4° C. and sonicated again for 10 minutes before each use.

The acceptor particles are biotinylated low density lipoproteins (LDL-biot). These particles are supplied by the company Amersham.

The reaction mixture is prepared in untreated ½ well white plates (Corning Costar) by addition, in the following order, of: 5 μl of HEPES 50 mM, NaCl 150 mM, BSA 0.1% (w/v), 0.05% sodium azide (w/v), pH 7.4 buffer; 5 μl of liposomes; 5 μl of LDL-biot; 5 μl of test products in DMSO; 5 μl of MTP. After incubation for 18-24 hours at 37° C., the reaction is stopped by adding 100 μl of Amersham SPA (Scintillation Proximity Assay) beads coupled to streptavidin, and the radioactivity is counted using a Top Count (Packard) at least one hour later. The inhibition of the transfer of the triglycerides with a compound is reflected by a reduction in the transferred radioactivity. The percentage of inhibition for a given compound is determined relative to controls that do not comprise compounds in the reaction mixture.

The results are expressed in terms of the IC₅₀, i.e. the concentration that allows a 50% inhibition of MTP. These results are summarised in the table below for a number of representative compounds of the invention.

TABLE

| Example | IC₅₀ (nM) |
|---|---|
| Example 33 | 230 |
| Example 40 | 220 |
| Example 42 | 270 |
| Example 48 | 340 |
| Example 49 | 136 |
| Example 90 | 44 |
| Example 91 | 223 |
| Example 93 | 61 |
| Example 94 | 193 |
| Example 98 | 312 |

Analysis of the secretion of apo B in the HepG2 human cell line:

The activity of a compound according to the invention can be evaluated by measuring the inhibition of apo B secretion in HepG2 cells.

The HepG2 cells (ECACC—No. 85011430) are used as model in the study of the in vitro hepatic secretion of lipoproteins (Dixon J. and Ginsberg H.-J. Lipid. Res.—1993, 34:167-179).

The HepG2 cells are cultured in Dulbecco's modified Eagle's medium comprising 10% foetal calf serum (DMEM and FBS-Gibco) in 96-well plates under an atmosphere of 5% carbon dioxide for 24 hours (about 70% confluence).

The test compounds are dissolved at a concentration of 2 or 10 mM in dimethyl sulfoxide (DMSO). Serial dilutions (1:3.16) are made in DMSO and are added (1:200—Robot Multimek Beckman) to the growth medium (200 microlitres) and then finally incubated for 24 hours in the various wells containing the HepG2 cells.

The 24-hour culture supernatant diluted to 1:5 (phosphate-buffered saline: PBS comprising 1% bovine serum albumin) is tested according to a sandwich-ELISA method specific for human apo B.

The results are expressed in terms of IC₅₀, i.e. the concentration that produces a 50% inhibition of apo B secretion in the HepG2 cells.

These results are collated in the table below for a number of representative compounds of the invention.

TABLE

| Example | IC₅₀ (nM) |
|---|---|
| Example 32 | 97 |
| Example 33 | 68 |
| Example 41 | 129 |
| Example 49 | 302 |
| Example 51 | 72 |
| Example 53 | 195 |
| Example 20 | 65 |
| Example 21 | 197 |
| Example 69 | 288 |
| Example 57 | 219 |
| Example 90 | 30 |
| Example 91 | 213 |
| Example 93 | 65 |
| Example 94 | 66 |
| Example 95 | 24 |
| Example 96 | 13 |
| Example 99 | 86 |

The examples that follow illustrate the present invention in greater detail.

The nuclear magnetic resonance spectra are the proton spectra, acquired at 300 MHz, and at ambient temperature. The chemical shifts are expressed in ppm and their reference is taken in each case on the signal of the deuterated solvent (chloroform at 7.25 ppm or dimethyl sulfoxide at 2.5 ppm).

The signals are described by the following abbreviations: s=singlet, d=doublet, t=triplet, dd=doublet of doublets, dt=doublet of triplets, td=triplet of doublets, m=multiplet, sept=septet.

The mass spectra are acquired using an LC/MS Platform-LC machine from Waters/Micromass in positive electrospray mode with a cone tension of 20 volts.

m.p. denotes the melting point.

MS denotes the mass spectrometry data.

NMR denotes the nuclear magnetic resonance data.

Preparation 1

### Preparation of 4-acetoxyethyl-1-bromobenzene

2.0 ml (28.1 mmol) of acetyl chloride are added to an ice-cooled solution of 3.76 g (18.7 mmol) of 4-bromophenethyl alcohol and 5.2 ml (37.3 mmol) of triethylamine in dichloromethane. The reaction mixture is stirred for 1 hour and then diluted in diethyl ether. The organic phase is washed with 1N HCl (twice), with saturated aqueous NaHCO₃ solution and then with saturated aqueous salt solution, after which it is dried over anhydrous magnesium sulfate and concentrated under reduced pressure. The crude product obtained (4.56 g, 100%) is pure and requires no further purification.

Preparation 2

### Preparation of 4′-acetoxyethylbiphenyl-2-carboxaldehyde

590 mg (1.11 mmol) of Pd(PPh₃)₄ are added under nitrogen to a mixture of 4.56 g (18.7 mmol) 4-bromophenethyl acetate, 2.56 g (17.1 mmol) of 2-formylbenzeneboronic acid and 7.78 g (51.2 mmol) of caesium fluoride in 86 ml of 1,2-dimethoxyethane. The resulting mixture is heated at 90° C. overnight. After cooling, the reaction mixture is diluted with water and extracted three times with diethyl ether. The combined mixture of the various extracted fractions is washed with water and then with saturated aqueous salt solution, after which it is dried over anhydrous magnesium sulfate and concentrated under reduced pressure. The crude product is purified by flash chromatography (ethyl acetate/hexane) to give 1.97 g (50%) of pure product.

Preparation 3

### Preparation of 4′-acetoxyethylbiphenyl-2-carboxylic acid

11.2 ml of a solution of Jones' reagent prepared by dissolving 35 g of CrO₃ in 98% H₂SO₄ (31.6 ml) in 100 ml of water are added dropwise to a solution of 4.0 g (14.9 mmol) of the aldehyde prepared in preparation 2 above, in 75 ml of acetone at 0° C. The reaction mixture is stirred at room temperature for six hours. The reaction medium is then diluted with diethyl ether and filtered through silica gel (washing with ether). The filtrate is concentrated under reduced pressure. The residue is taken up in diethyl ether and washed twice with water and with saturated aqueous salt solution, after which it is dried over anhydrous magnesium sulfate and concentrated under reduced pressure. The yield is 2.77 g (65%).

Preparation 4

### Preparation of 4-(1-hydroxy-2-methylpropyl)-1-bromobenzene

20 ml of a 2M solution of isopropylmagnesium chloride in tetrahydrofuran (40 mmol) are added dropwise to a solution of 7.4 g (40 mmol) of 4-bromobenzaldehyde in 20 ml of diethyl ether at −78° C., maintained under nitrogen. Once the addition is complete, the reaction mixture is maintained at −78° C. with stirring for two hours, after which the reaction is stopped by adding saturated ammonium chloride solution. The reaction mixture is left at room temperature, until this room temperature is reached, after which the aqueous phase is extracted three times with diethyl ether. The combined ether fractions are then washed with 1N HCl, with water and with saturated aqueous salt solution, after which the resulting solution is dried over anhydrous magnesium sulfate and concentrated under reduced pressure. 8.33 g of crude product are obtained in a purity of 60%, the remainder consisting of the starting aldehyde.

Preparation 5

### Preparation of 4-(1-oxo-2-methylpropyl)-1-bromobenzene

15.4 ml of a solution of Jones' reagent, prepared by dissolving 35 g of CrO₃ in 98% H₂SO₄ (31.6 ml), in 100 ml of water are added to a solution of 4.7 g (about 20.5 mmol) of the alcohol obtained in preparation 4 (in a purity of 60%, as results from the reaction for carrying out preparation 4) in 61 ml of acetone.

After two hours, an analysis by thin layer chromatography shows that all the starting material has been consumed. The reaction medium is then filtered and concentrated under reduced pressure. The residue is taken up diethyl ether, washed with 1N NaOH (twice), with water and with saturated aqueous salt solution. The organic phase is then dried over anhydrous magnesium sulfate and concentrated under reduced pressure to give 2.85 g (about 46% of 4-bromobenzaldehyde). The product is pure enough to be used without further purification.

Preparation 6

### Preparation of 4′-(1-oxo-2-methylpropyl)biphenyl-2-carboxaldehyde

385 mg (0.33 mmol) of Pd(PPh₃)₄ are added to a solution of 2.78 g (12.25 mmol) of the bromo ketone obtained in preparation 5 above, 1.67 g (11.15 mmol) of 2-formylbenzeneboronic acid and 5.07 g (33.37 mmol) of caesium fluoride in 56 ml of 1,2-dimethoxyethane maintained under nitrogen. The resulting mixture is heated at 90° C. overnight. After cooling, the reaction mixture is diluted with water and is extracted three times with diethyl ether. The combined extracted fractions are washed with water and then with saturated aqueous salt solution, after which they are dried over anhydrous magnesium sulfate and concentrated under reduced pressure. The crude product is purified by flash chromatography (ethyl acetate/hexane) to give 1.56 g (55%) of pure product.

Preparation 7

### Preparation of 4′-(1-oxo-2-methylpropyl)biphenyl-2-carboxylic acid

5 ml of a solution of Jones' reagent, prepared by dissolving 35 g of CrO₃ in 98% H₂SO₄ (31.6 ml) in 100 ml of water, are added to a solution of 1.3 g (5.15 mmol) of the aldehyde obtained in preparation 6 dissolved in 20 ml of acetone, maintained at 0° C. The reaction medium is stirred at room temperature overnight. The reaction mixture is concentrated under reduced pressure and the residue is taken up in diethyl ether and filtered through silica gel (washing with diethyl ether). The filtrate is extracted with 1N NaOH. The basic fractions are then acidified with 1N HCl and extracted three times with ethyl acetate. The ethyl acetate fractions are washed with water and then with saturated aqueous salt solution, after which the resulting solution is dried over anhydrous magnesium sulfate and concentrated under reduced pressure. The yield is 1.40 g (100%).

Preparation 8

### Preparation of 4-ethylaminocarbonyloxyethyl-1-bromobenzene

2.0 g (9.95 mmol) of p-bromophenethyl alcohol are dissolved in 15 ml of dichloromethane and 1.73 ml (1.29 g, 9.95 mmol) of N,N-diisopropylethylamine are added thereto. The reaction medium is treated with 1.42 g (19.9 mmol) of ethyl isocyanate in 5 ml of dichloromethane and then heated at 40° C. overnight. The reaction medium is then cooled to room temperature, diluted with dichloromethane and then washed with water and with saturated aqueous salt solution. The organic phase is dried over anhydrous magnesium sulfate and concentrated under reduced pressure to give 3.13 g of crude product. This crude product is purified by chromatography on a column of silica using as eluent a 1:1 mixture of ethyl acetate and petroleum ether, so as to give a pure colourless oil (1.64 g, i.e. 61% yield).

Preparation 9

### Preparation of 4′-ethylaminocarbonyloxyethylbiphenyl-2-carboxaldehyde

0.203 g (0.176 mmol) of Pd(PPh₃)₄ is added to a solution of 1.23 g (8.23 mmol) of benzaldehyde-2-boronic acid, 1.60 g (5.88 mmol) of the bromide obtained in preparation 8 and 2.66 g (17.5 mmol) of caesium fluoride in 22 ml of 1,2-dimethoxyethane. The solution is then heated at 85° C. overnight under nitrogen. The reaction medium is cooled to room temperature and then diluted with diethyl ether and washed three times with water. The organic phase is dried over anhydrous magnesium sulfate and concentrated under reduced pressure. The crude product (1.96 g) is purified by chromatography on a column of silica using as eluent an ethyl acetate/hexane mixture in a 1:4 ratio. The product is obtained in the form of a pale yellow oil (1.35 g: a yield of 77%).

Preparation 10

### Preparation of 4′-ethylaminocarbonyloxyethylbiphenyl-2-carboxylic acid

1.34 g (4.51 mmol) of the aldehyde obtained in preparation 9 are dissolved in 11 ml of acetone and cooled to 0° C. 3 ml of a Jones' reagent, prepared by dissolving 35 g (350 mmol) of chromium trioxide dissolved in 98% sulfuric acid (31.6 ml) in 100 ml of water, are added dropwise and the solution is then stirred at 0° C. for 1 hour. The reaction medium is then left stirring overnight at room temperature. At this point, the chromium salts precipitated from the reaction medium. The solution is filtered through silica, the product being entrained by washing with ethyl acetate. The organic phase is extracted with 1M NaOH, so as to entrain the product in the aqueous phase, leaving the impurities in the organic phase. The aqueous phase is then acidified with 1M HCl and the product is extracted with ethyl acetate. The organic phase is dried over anhydrous magnesium sulfate and concentrated under reduced pressure. The product is purified by chromatography on a column of silica, using as eluent a mixture of ethyl acetate and petroleum ether in a 2:1 ratio. A colourless solid is thus obtained (1.0 g: a yield of 71%).

Preparation 11

### Preparation of 3-nitro-6-allyloxyphenol

7.59 g (54.9 mmol) of K₂CO₃, and then 4.7 ml (54.3 mmol) of allyl bromide are added to a solution of 7.52 g (48.5 mmol) of 4-nitrocresol in 125 ml of N,N-dimethylformamide. The resulting reaction medium is stirred at room temperature overnight, and then diluted with water and extracted twice with diethyl ether. The traces of starting cresol thus remain in the basic aqueous phase. The combined ether fractions (comprising a mixture of the desired monoallyl derivative and of the bisallyl derivative) are then extracted twice with 1N NaOH. The ether phase is then discarded and the combined basic fractions are acidified to pH 3 with 2N HCl. The aqueous phase is then extracted three times with ether. The combined ether fractions are then washed with water and with saturated aqueous salt solution, after which the resulting solution is dried over anhydrous magnesium sulfate and concentrated under reduced pressure. 4.18 g (44%) of the desired monoallyl derivative are obtained, this product being pure enough to be used without further purification.

Preparation 12

### Preparation of 3-nitro-6-allyloxyphenol epoxide

6.8 g (23.6 mmol) of 60% meta-chloroperbenzoic acid are added to a solution of 4.18 g (21.4 mmol) of the allylic compound obtained in preparation 11 in 42 ml of dichloromethane. The reaction medium is stirred overnight at room temperature, until an analysis by thin layer chromatography indicates total consumption of the starting material. The reaction mixture is then diluted with diethyl ether and extracted three times with saturated sodium bicarbonate solution. The organic phase is then washed with water and then with saturated aqueous salt solution, dried over anhydrous magnesium sulfate and then concentrated under reduced pressure. The crude reaction medium is then triturated with diethyl ether so as to eliminate the excess meta-chloroperbenzoic acid and its by-products, to give 2.68 g (59%) of the desired epoxide, which is pure enough to be used without further purification.

Preparation 13

### Preparation of 7-nitro-2-hydroxymethylbenzodioxane

2.75 g (50.9 mmol) of sodium methoxide are added to a solution of 2.68 g (12.7 mmol) of the epoxide obtained in preparation 12 in 50 ml of methanol. The resulting reaction mixture is stirred at room temperature overnight and then concentrated under reduced pressure. The residue is diluted with water and extracted three times with ethyl acetate. The combined fractions are washed with 1N NaOH, with water and with saturated aqueous salt solution and then dried over anhydrous magnesium sulfate and concentrated under reduced pressure. The resulting product, 1.92 g (72%), is used in the next reaction without further purification.

Preparation 14

### Preparation of 7-nitro-2-tert-butyldimethylsilyloxymethylbenzodioxane

2.09 g (7.5 mmol) of a solution of t-butyldimethylsilyl chloride in 20 ml of dichloromethane are added to a solution of 1.06 g (5 mmol) of the alcohol obtained in preparation 13 in 30 ml of dichloromethane comprising 2.6 ml (10 mmol) of triethylamine and 61 mg (0.5 mmol) of 4-dimethylaminopyridine. The reaction medium is left stirring at room temperature overnight and then diluted with diethyl ether, washed with 1N HCl, with saturated sodium bicarbonate solution, with water and with saturated aqueous salt solution, and then dried over anhydrous magnesium sulfate and concentrated under reduced pressure. The resulting crude product is purified by flash chromatography so as to give the desired product (1.4 g, i.e. 86% yield).

Preparation 15

### Preparation of 7-amino-2-tert-butyldimethylsilyloxymethylbenzodioxane

1.4 g (4.3 mmol) of the nitrobenzodioxane obtained in preparation 14 are dissolved in 25 ml of ethanol. 200 mg of 10% palladium-on-charcoal are added to the reaction medium and the resulting mixture is stirred overnight under a hydrogen atmosphere (40 psi). The catalyst is then removed by filtration and the reaction medium is concentrated under reduced pressure, to give the expected aniline, which is purified by flash chromatography using as eluent a mixture of diethyl ether and hexane. The yield obtained is 89% (1.14 g).

Preparation 16

### Preparation of 7-nitro-2-methoxymethylbenzodioxane

1.05 g (5 mmol) of the alcohol obtained in preparation 13 in 1.5 ml of N,N-dimethylformamide are added to a suspension of 400 mg (10 mmol) of sodium hydride (at 60% in oil), washed with hexane, in 1 ml of N,N-dimethylformamide. After stirring at room temperature for one hour, 1.05 g (5 mmol) of methyl iodide are added. The resulting reaction mixture is left stirring at room temperature overnight, and the reaction is then quenched by slow addition of water. The reaction medium is extracted three times with ethyl acetate. The organic fractions obtained are washed with water and then with saturated aqueous salt solution, after which the resulting solution is dried over anhydrous magnesium sulfate and concentrated under reduced pressure. 1.08 g (96% yield) of the crude product are thus obtained, which product is then used without further purification.

Preparation 17

### Preparation of 7-amino-2-methoxymethylbenzodioxane

531 mg (2.35 mmol) of the nitrobenzodioxane obtained in preparation 16 are dissolved in 20 ml of ethanol. 140 mg of 10% palladium-on-charcoal are added to the reaction medium and the mixture is left stirring overnight under a hydrogen atmosphere (40 psi). The catalyst is then removed by filtration and the reaction medium is concentrated under reduced pressure, to give the expected aniline, which is purified by flash chromatography using a mixture of diethyl ether and hexane as eluent. The resulting yield is 419 g (91%).

Preparation 18

### Preparation of 4′-isopropylbiphenyl-2-carboxylic acid

Step a

### 4′-Isopropylbiphenyl-2-carboxaldehyde

A mixture of 30.0 g (0.20 mol) of 2-formylphenylboronic acid, 43.8 g (0.22 mol) of 4-bromoisopropylbenzene, 91.0 g (0.60 mol) of caesium fluoride and 6.9 g (0.0060 mol) of tetrakis(triphenylphosphine)palladium(0) in 700 ml of 1,2-dimethoxyethane is refluxed under a nitrogen atmosphere for 3 hours. After cooling, 1.5 l of diethyl ether and 1 l of water are added to the reaction medium and the organic phase is separated out, dried over sodium sulfate and concentrated. The oily residue obtained is purified by chromatography on a column of silica (eluent: 30/1 hexane/ethyl acetate) to give 25.3 g (56.4%) of a pale yellow oil corresponding to the title compound.

NMR: (CDCl₃) δ (ppm): 1.31 (6H, d, J=7 Hz); 2.99 (1H, sept, J=7 Hz); 7.26-7.39 (4H, m); 7.41-7.53 (2H, m); 7.54-7.69 (1H, m); 7.93-8.13 (1H, m); 10.01 (1H, s).

IR:

v (C=0): 1694 cm⁻¹

Step b

### 4′-Isopropylbiphenyl-2-carboxylic acid

A solution of 23.7 g (0.15 mol) of potassium permanganate in 500 ml of water is added over 15 minutes to a solution at room temperature of 22.4 g (0.10 mol) of 4′-isopropylbiphenyl-2-carboxaldehyde in 500 ml of acetone. The temperature of the reaction medium rises to 32° C. and this medium is stirred for 4 hours at room temperature. After addition of a sodium thiosulfate solution and acidification with 10 N hydrochloric acid, the solution obtained is extracted with 2×500 ml of dichloromethane. This organic solution is extracted with N sodium hydroxide and the basic solution is then washed with diethyl ether, neutralised with N hydrochloric acid and extracted with dichloromethane. These organic extracts are dried over sodium sulfate and concentrated, and the residue obtained is purified by chromatography on a column of silica (eluent: 2/1 hexane/ethyl acetate) to give 5.8 g (24.1%) of a solid corresponding to the title compound.

NMR: (CDCl₃) δ (ppm): 1.29 (6H, d, J=7 Hz); 2.94 (1H, sept, J=7 Hz); 7.18-7.33 (4H, m); 7.34-7.45 (2H, m); 7.48-7.61 (1H, m); 4.85-8.05 (1H, m).

The following intermediate acids are prepared in the same manner:
3′-(trifluoromethyl)biphenyl-2-carboxylic acid,
3′,4′-dimethylbiphenyl-2-carboxylic acid,
4′-(trifluoromethoxy)biphenyl-2-carboxylic acid,
4′-isopropylbiphenyl-2-carboxylic acid.
   Preparation 19

### Preparation of 6-methyl-4′-trifluoromethoxy)biphenyl-2-carboxylic acid

Step a

### Methyl 2-bromo-3-methylbenzoate

A mixture of 3.3 g (15.3 mmol) of 2-bromo-3-methylbenzoic acid and 2.9 g (15.3 mmol) of p-toluenesulfonic acid in 77 ml of methanol is refluxed overnight. After cooling, the reaction medium is concentrated under reduced pressure and the residue is taken up in diethyl ether, washed twice with saturated sodium hydrogen carbonate solution, then with water and finally with brine. The organic solution is dried over anhydrous magnesium sulfate and concentrated under reduced pressure. The crude substance (3.43 g, yield=96%) is used in the next step without further purification.

Step b

### Methyl 6-methyl-4′-(trifluoromethoxy)biphenyl-2-carboxylate

25 ml (10.0 mmol) of aqueous 0.4 M sodium carbonate solution and then 303 mg (0.26 mmol) of tetrakis(triphenylphosphine)palladium(0) are added to a solution of 2.0 g (8.7 mmol) of methyl 2-bromo-3-methylbenzoate and 2.16 g (10.5 mmol) of 4-(trifluoromethoxy)boronic acid in 25 ml of acetonitrile. The mixture is refluxed overnight; after cooling, the reaction medium is diluted with 50 ml of water and extracted with diethyl ether. The combined organic extracts are washed twice with water, dried over anhydrous magnesium sulfate and concentrated under reduced pressure. The residue is purified by flash chromatography (eluent: 5% of diethyl ether in hexane) to give 2.4 g (89%) of the title compound.

Step c

### 6-Methyl-4′-(trifluoromethoxy)biphenyl-2-carboxylic acid

A solution of 8.5 ml (17.03 mmol) of 2 N sodium hydroxide is added with stirring to a solution of 2.4 g (7.74 mmol) of methyl 6-methyl-4′-(trifluoromethoxy)-biphenyl-2-carboxylate in 40 ml of methanol, and the reaction medium is then maintained at 60° C. for 3 hours. After addition of a further 5.0 ml (10.02 mmol) of 2 N sodium hydroxide, heating is continued at 60° C. overnight. After cooling, the reaction medium is concentrated under reduced pressure; the residue is taken up in 150 ml of water, acidified with 2 N hydrochloric acid and extracted twice with ethyl acetate. The combine extracts are washed with water, dried over anhydrous magnesium sulfate and concentrated under reduced pressure. The white solid obtained is dried in a vacuum oven to give 2.1 g (92%) of the title compound.

The following intermediate acid is prepared in the same manner: 4′-ethyl-6-methylbiphenyl-2-carboxylic acid.

### EXAMPLES

Typical procedure for the preparation of a compound of the formula I from a carboxylic acid containing a free —COOH function, of the formula II and an amine of the formula III.

A solution of the carboxylic acid (0.2 mmol) in 0.3 ml of a volumetric mixture in a 1:9 ratio of N,N-diisopropylethylamine and N,N-dimethylformamide is added to a solution of the amine (0.2 mmol) in the same mixture (0.3 ml). A further volume of 0.03 ml of N,N-diisopropylethylamine is then added, followed by addition of a solution of O-(benzotriazol-1-yl)-N,N,N′,N′-tetramethyluronium hexafluorophosphate (HBTU) (0.24 mmol) in 0.3 ml of N,N-dimethylformamide. The mixture is stirred at room temperature overnight. After evaporating off the solvent, the mixture is dissolved in dichloromethane and washed successively with three times 1 ml of aqueous potassium carbonate solution (7% weight/volume) and with 1 ml of water. After analysis by LC/MS, the solvent is evaporated to dryness.

Other solvents that can be used instead of N,N-dimethylformamide: dichloromethane and acetonitrile.

Other coupling reagents that can be used instead of HBTU: O-(7-azabenzotriazol-1-yl)-N,N,N′,N′-tetramethyluronium hexafluorophosphate, 1-ethyl-3-(3′-dimethylaminopropyl)carbodiimide hydrochloride, isobutyl chloroformate, methanesulfonyl chloride, bromotris(pyrrolidino)phosphonium hexafluorophosphate, chloro-N,N,N′,N′-bis(tetramethylene)formamidinium tetrafluoroborate.

Other bases that can be used instead of N,N-diisopropylethylamine: 4-dimethylaminopyridine, triethylamine, N-methylmorpholine. Typical procedure for the preparation of a compound of the formula I from a carboxylic acid chloride (activated derivative of a carboxylic acid of the formula II) and an amine of the formula III

Step a

### Preparation of Carboxylic Acid Chlorides from Carboxylic Acids

2-3 drops of a 30% solution of N,N-dimethylformamide in dichloromethane are added to a mixture of 1.05 mmol of carboxylic acid and 0.18 ml (2.06 mmol) of oxalyl chloride in 5 ml of dichloromethane. The resulting mixture is stirred at 0° C. for 1.5 hours; it is then diluted with 3.5 ml of anhydrous dichloromethane and used immediately in the next step without further treatment.

Step b

### Preparation of Carboxamides from Carboxylic Acid Chlorides and Amines

26 μl (0.15 mmol) of N,N-diisopropylethylamine and 550 μl (0.165 mmol) of the acid chloride solution obtained above are successively added to 550 μl (0.15 mmol) of a solution of amine in anhydrous dichloromethane. The mixture is stirred overnight at room temperature and is then concentrated to dryness under reduced pressure. The residue is dissolved in 1.2 ml of dichloromethane and 2 ml of aqueous 0.5 N sodium hydrogen carbonate solution are added. The organic phase is washed successively with twice 800 μl of water, with 800 μl of 0.5 N hydrochloric acid and with 800 μl of water. After analysis by LC/MS, the solvent is evaporated to dryness.

Other solvents that can be used instead of dichloromethane: N,N-dimethylformamide and acetonitrile.

Specific procedures for the preparation of a number of examples of the invention are given below by way of illustration.

### Example 17

A solution of 895 mg (3.46 mmol) of 4′-isopropylbiphenyl-2 carboxylic acid in 23 ml of acetonitrile is added to a solution of 941 mg (3.18 mmol) of the amine obtained in preparation 15 in 20 ml of acetonitrile comprising 650 μl (4.66 mmol) of triethylamine and 42 mg (0.35 mmol) of 4-dimethylaminopyridine, with stirring. Stirring is continued at room temperature overnight. The reaction mixture is then diluted with ethyl acetate and the organic phase is washed with 1N HCl, with saturated aqueous sodium bicarbonate solution, with water and with saturated aqueous salt solution. The reaction medium is then dried over anhydrous magnesium sulfate and concentrated under reduced pressure. Purification by flash chromatography using an ethyl acetate/hexane mixture gives 1.60 g (95%) of the pure expected product.

### Example 18

10.3 ml (10.3 mmol) of a 1M solution of tetrabutylammonium fluoride in tetrahydrofuran are added to a solution of 1.6 g (3.01 mmol) of the compound of Example 17 in 41 ml of tetrahydrofuran. The reaction medium is concentrated under reduced pressure. The residue is taken up in ethyl acetate and washed with water and then with saturated aqueous salt solution, after which the resulting solution is dried over anhydrous magnesium sulfate and concentrated under reduced pressure. The pure product is obtained by purification by flash chromatography, using a mixture of ethyl acetate and hexane as eluent. The yield is 91%. 1.11 g of pure product are obtained.

### Example 19

2 ml of acetic anhydride are added to a solution of 105 mg (0.26 mmol) of the alcohol prepared in Example 18 in 4 ml of pyridine. After reaction overnight at room temperature, the volatile substances are removed by evaporation under reduced pressure, the residue being treated azeotropically with toluene. Purification by flash chromatography gives the expected acetate in pure form: 105 mg (91% yield).

### Example 20

A solution of 4′-isopropylbiphenyl-2-carboxylic acid (218 mg; 0.84 mmol) in 4 ml of acetonitrile is added to a solution of 137 mg (0.7 mmol) of the amine obtained in preparation 17, in 3 ml of acetonitrile comprising 195 μl (1.4 mmol) of triethylamine and about 10 mg (0.08 mmol) of 4-dimethylaminopyridine, with stirring. The reaction mixture is stirred at room temperature overnight. The reaction mixture is then diluted with ethyl acetate and the organic phase is washed with 1N HCl, with saturated aqueous sodium bicarbonate solution, with water and with saturated aqueous salt solution. The reaction medium is then dried over anhydrous magnesium sulfate and concentrated under reduced pressure. The crude product is purified by flash chromatography using an ethyl acetate/hexane mixture as eluent, to give 172 mg (59%) of the pure expected product.

### Example 21

39 μl (0.497 mmol) of ethyl isocyanate are added to a solution of 167 mg (0.416 mmol) of the alcohol obtained in Example 18 in 8 ml of anhydrous dichloromethane comprising 108 μl (0.621 mmol) of diisopropylethylamine. The reaction mixture is heated overnight at 40° C. After cooling, the reaction medium is diluted with ethyl acetate. The organic phase is washed twice with 1N HCl, with saturated sodium bicarbonate solution, with water and with saturated aqueous salt solution, and then dried over anhydrous magnesium sulfate and concentrated under reduced pressure. The crude reaction medium (which comprises a small amount of starting material) is purified by flash chromatography using as eluent an ethyl acetate/hexane mixture. The yield is 69% (136 mg).

### Example 22

A solution of the alcohol obtained in Example 18 (110 mg; 0.27 mmol) in 1.5 ml of N,N-dimethylformamide is added to a suspension of 21.6 mg (0.54 mmol) of sodium hydride at 60% in oil, washed with hexane, in 1 ml of N,N-dimethylformamide. After reaction for one hour at room temperature with stirring, 25 μl (0.40 mmol) of methyl iodide are added. The resulting reaction mixture is stirred at room temperature overnight, and the reaction is then quenched by slow addition of water. The reaction mixture is extracted three times with ethyl acetate. The combined organic fractions are washed with water and with saturated aqueous salt solution, and then dried over anhydrous magnesium sulfate and concentrated under reduced pressure. Analysis by LCMS indicates the presence of the bis-methyl product along with a small amount of the monomethyl product (which is assumed to be the product methylated on the nitrogen of the amide). Specifically, the monomethyl product obtained in Example 20 has the same retention time, R_{f}, as the starting alcohol, and no trace of it is observed. The bismethyl product is isolated by flash chromatography using as eluent a mixture of ethyl acetate and hexane. A yield of 61% is obtained, i.e. 71 mg.

### Example 27

30 μl (0.48 mmol) of methyl iodide are added to a solution of 132 mg (0.324 mmol) of the alcohol obtained in Example 18 in 6.4 ml of acetonitrile. The reaction mixture is stirred overnight at room temperature, and then diluted with ethyl acetate. The organic phase is washed twice with 1N HCl, with saturated aqueous sodium bicarbonate solution, with water and with saturated aqueous salt solution, and then dried over anhydrous magnesium sulfate and concentrated under reduced pressure. The crude product, 132 mg (98%) is pure enough to be used in the next reaction without further purification.

### Example 29

2 ml of acetic anhydride are added to a solution of 57 mg (0.137 mmol) of the alcohol obtained in Example 27. After reaction overnight at room temperature, the volatile substances are removed by evaporation under reduced pressure, the residue being treated azeotropically with toluene. Purification by flash chromatography gives the pure acetate (58 mg; 92% yield).

### Example 33

### Preparation of (4′-isopropylbiphen-2-yl)-N-(2,2-difluorobenzo[1,3]dioxol-5-yl)carboxamide

A solution of 4.57 g (0.036 mol) of oxalyl chloride in 10 ml of dichloromethane is added to a mixture, maintained between 0 and 5° C., of 4.8 g (0.020 mol) of 4′-isopropylbiphenyl-2-carboxylic acid in 50 ml of dichloromethane, followed by addition of 2 drops of N,N-dimethylformamide. The resulting mixture is stirred for 3 hours at room temperature and then concentrated under reduced pressure to give the 4′-isopropylbiphenyl-2-carboxylic acid chloride. A solution of this acid chloride in 30 ml of dichloromethane is added at between 0 and 5° C. to a solution of 3.4 g (0.196 mol) of 2,2-difluoro-5-aminobenzodioxole and 5.3 g of triethylamine (0.0524 mol) in 50 ml of dichloromethane. After stirring for 3 hours at room temperature, aqueous sodium bicarbonate solution is added. The organic phase is washed with water, dried over sodium sulfate and concentrated to dryness under reduced pressure to give a solid, which is purified by recrystallisation from 150 ml of heptane, followed by chromatography on a column of silica (eluent: 2/1 hexane/ethyl acetate) and a further recrystallisation from a mixture of 70 ml of heptane and 20 ml of ethyl acetate. 3.0 g (38.7%) of a white powder corresponding to the title compound are obtained.

¹H NMR: (CDCl₃) δ (ppm): 1.28 (6H, d, J=7 Hz); 2.97 (1H, sept, J=7 Hz); 6.24-6.38 (1H, m); 6.73-6.91 (2H, m); 7.11-7.21 (1H, m); 7.28-7.62 (7H, m); 7.83-7.98 (1H, m).

### Example 52

664 mg (1.75 mmol) of O-(7-azabenzotriazol-1-yl)-N,N,N′,N′-tetramethyluronium hexafluorophosphate (HBTU) are added to a solution of 415 mg (1.46 mmol) of 4′-acetoxyethylbiphenyl-2-carboxylic acid and 303 mg (1.75 mmol) of 5-amino-2,2-difluorobenzodioxole in 14.6 ml of acetonitrile comprising 381 μl (2.19 mmol) of diisopropylethylamine. The reaction mixture is stirred at room temperature overnight and then diluted with ethyl acetate. The organic phase is washed with 1N HCl, with saturated sodium bicarbonate solution, with water and with saturated salt solution, and then dried over anhydrous magnesium sulfate and concentrated under reduced pressure. The crude product is purified by flash chromatography using an ethyl acetate/hexane mixture as eluent, to give 391 g (61%) of pure product.

### Example 53

1.58 g of solid potassium carbonate are added to a solution of 500 mg (1.14 mmol) of the acetate obtained in Example 52 in 10 ml of aqueous methanol (10% water). The resulting reaction mixture is stirred overnight at room temperature and then concentrated under reduced pressure. The residue is taken up in ethyl acetate and washed with water. The organic phase is then dried over anhydrous magnesium sulfate, filtered and concentrated under reduced pressure to give 450 mg (99%) of the expected product, which can be used in this form without further purification.

### Example 54

2.37 g (6.24 mmol) of HBTU are added to a solution of 1.4 g (5.2 mmol) of 4′-isopropylcarbonylbiphenyl-2-carboxylic acid and 1.08 g (6.2 mmol) of 5-amino-2,2-difluorobenzodioxole in 52 ml of acetonitrile comprising 1.36 ml (7.81 mmol) of diisopropylethylamine. The reaction mixture is stirred at room temperature for three days and then diluted with ethyl acetate. The organic phase is washed with 1N HCl, with saturated sodium bicarbonate solution, with water and with saturated aqueous salt solution, and then dried over anhydrous magnesium sulfate and concentrated under reduced pressure. The crude product is purified by flash chromatography using an ethyl acetate/hexane mixture as eluent. The product thus obtained is purified after taking up in ether and washing twice with 10% potassium carbonate solution, with water and with saturated aqueous salt solution. The organic phase is dried over anhydrous magnesium sulfate and concentrated under reduced pressure to finally give 1.62 g (74%) of the pure expected product.

### Example 55

357 mg (9.44 mmol) of sodium borohydride are added a solution of 1 g (2.36 mmol) of the ketone obtained in Example 54 in 24 ml of aqueous methanol (10% water). The reaction mixture is stirred for two hours at room temperature and then diluted with diethyl ether. The organic phase is washed twice with 1N HCl, with saturated aqueous sodium bicarbonate solution, with water and with saturated aqueous salt solution, and then dried over anhydrous magnesium sulfate and concentrated under reduced pressure. The crude product, 775 mg (77%), is used in this form in the next reaction without further purification.

### Example 69

A solution of 403 mg (0.95 mmol) of the alcohol obtained in Example 55 in 20 ml of toluene comprising 40 mg (10% v/v) of para-toluenesulfonic acid is heated at 80° C. overnight. After cooling, the reaction mixture is diluted with diethyl ether, washed three times with saturated aqueous sodium bicarbonate solution, with water and with saturated aqueous salt solution. The organic phase is then dried over anhydrous magnesium sulfate and concentrated under reduced pressure. The crude product, 295 mg (76%), is used in this form in the next reaction without further purification.

### Example 57

A solution of 267 mg (0.66 mmol) of the olefin obtained in Example 69 in 15 ml of ethanol comprising 42 mg of 10% palladium-on-charcoal is stirred overnight under a hydrogen atmosphere (30 psi). The catalyst is removed by filtration and the solvent is evaporated off under reduced pressure to give 198 mg (73%) of product, which is used in this form without further purification.

### Example 68

Step a

### Preparation of 5-[4′-mesyloxyethylbiphen-2-ylcarbonylamino]-2,2-difluorobenzodioxole

306 ml (2.2 mmol) of triethylamine are added to a solution of 450 mg (1.1 mmol) of the alcohol obtained in Example 53 in 5 ml of anhydrous dichloromethane at 0° C., followed by dropwise addition of 160 mg (108 ml; 1.4 mmol) of mesyl chloride. The reaction mixture is stirred at this temperature for 4 hours and then diluted with dichloromethane. The organic phase is washed with water, with 10% citric acid, with saturated aqueous sodium bicarbonate solution and with saturated aqueous salt solution. It is then dried over anhydrous magnesium sulfate and concentrated under reduced pressure. The crude product is purified by flash chromatography using a mixture of ethyl acetate and hexane as eluent, to give 450 mg (91%) of the expected mesylate.

Step b

### Preparation of the Compound of Example 68

29 mg (0.421 mmol) of imidazole are dissolved in 1.5 ml of acetonitrile and 2-tert-butylimino-2-diethylamino-1,3-dimethylperhydro-1,3,2-diazaphosphorine is added. The reaction mixture is stirred for 10 minutes, after which a solution of 200 mg (0.421 mmol) of the mesylate obtained in the above step in 1.5 ml of acetonitrile is added. The reaction mixture is stirred overnight at room temperature and then diluted with ethyl acetate. The organic phase is washed with 1N HCl, with saturated aqueous sodium bicarbonate solution, with water and with saturated aqueous salt solution, and then dried over anhydrous magnesium sulfate and concentrated under reduced pressure. An analysis by thin layer chromatography indicates the presence of a small amount of starting material together with a more polar product, which is isolated by flash chromatography, first using ethyl acetate as eluent, and then a mixture of methanol and ethyl acetate in 15:85 proportions. The yield obtained is 35% (70 mg).

Examples illustrating the invention are collated in Tables 1 to 8. They were prepared in accordance with the procedures described above, using the appropriate reagents.

TABLE 1

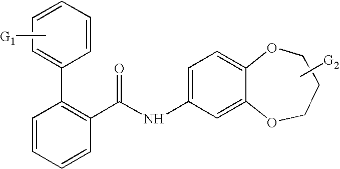

| Example | | | | |
|---|---|---|---|---|
| No. | G₁ | G₂ | NMR | MS |
| 1 | 4′-CF₃ | H | (DMSO-d6): 2.06 (2H, | a = 414.1 |
| | | | m); 4.06 (4H, m); 6.85 (1 | b = 436.1 |
| | | | H, m); 7.07 (1H, m); 7.19 | c = 412.1 |
| | | | (1H, m); 7.50-7.75 (6H, | |
| | | | m); 7.76 (2H, m); 10.26 | |
| | | | (1H, broad s). | |
| 2 | H | H | (DMSO-d6): 2.03 (2H, | a = 346.1 |
| | | | m); 4.03 (4H, m); 6.84 (1 | b = 368.1 |
| | | | H, m); 6.99 (1H, m); 7.15 | c = 344.1 |
| | | | (1H, m); 7.22-7.62 (9H, | |
| | | | m); 10.10 (1H, broad s). | |
| 3 | 4′-CH₃ | H | (DMSO-d6): 2.04 (2H, | a = 360.1 |
| | | | m), 2.27 (3H, s); 4.04 (4 | b = 382.1 |
| | | | H, m); 6.85 (1H, m); 7.03 | c = 358.1 |
| | | | (1H, m); 7.11-7.23 (3H, | |
| | | | m); 7.24-7.35 (2H, m); | |
| | | | 7.35-7.58 (4H, m); 10.12 | |
| | | | (1H, broad s). | |
| 4 | 4′-CH₂—CH₃ | H | (DMSO-d6): 1.15 (3H, t, | a = 374.1 |
| | | | J = 7.68 Hz); 2.04 (2H, | b = 396.1 |
| | | | m); 2.58 (2H, q, J = 7.68 | c = 372.1 |
| | | | Hz); 4.04 (4H, m); 6.85 | |
| | | | (1H, m); 7.02 (1H, m); | |
| | | | 7.13-7.25 (3H, m); 7.25- | |
| | | | 7.38 (2H, m); 7.38-7.63 | |
| | | | (4H, m); 10.09 (1H, | |
| | | | broad s). | |
| 5 | 4′-CH(CH₃)₂ | H | (DMSO-d6): 1.17 (6H, d, | a = 388.1 |
| | | | J = 9 Hz); 2.04 (2H, m); | b = 410.1 |
| | | | 2.87 (1H, sept, J = 9 | c = 386.1 |
| | | | Hz); 4.04 (4H .m); 6.85 | |
| | | | (1H, m); 7.03 (1H, m); | |
| | | | 7.14 (1H, m); 7.18-7.27 | |
| | | | (2H, m); 7.27-7.37 (2H, | |
| | | | m); 7.37-7.58 (4H, m); | |
| | | | 10.08 (1H, broad s). | |
| 6 | 4′-Cl | H | (DMSO-d6): 2.05 (2H, | a = 380 |
| | | | m); 4.06 (4H, m); 6.86 | b = 402 |
| | | | (1H, m); 7.06 (1H, m); | c = 378 |
| | | | 7.21 (1H, m); 7.35-7.80 | |
| | | | (8H, m); 10.18 (1H, | |
| | | | broad s) | |
| 7 | 3′-CF₃ | H | (DMSO-d6): 2.04 (2H, | |
| | | | m); 4.04 (4H, m); 6.84 (1 | a = 414.1 |
| | | | H, m); 6.99 (2H, m); | b = 436.1 |
| | | | 7.44-7.76 (8H, m); 10.19 | c = 412.1 |
| | | | (1H, broad s). | |

In this table, and also in the rest of the examples, a, b and c are defined as follows:
a corresponds to ES+M+H
b corresponds to ES+M+23

c corresponds to ES−M−H

TABLE 2

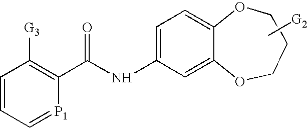

| Example | | | | | |
|---|---|---|---|---|---|
| No. | G₃ | G₂ | P₁ | NMR | MS |
| 8 | N-pyrrolyl | H | C | — | a = 335.1 |
| | | | | | b = 357.1 |
| | | | | | c = 333.1 |
| 9 | phenyl | H | N | — | a = 347.1 |
| | | | | | b = 369.1 |
| | | | | | c = 345.1 |

a, b and c being as defined above.

TABLE 3

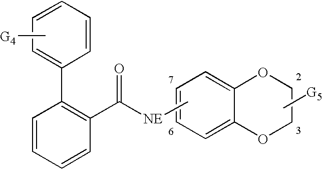

| Example | Bond to the | | | | | |
|---|---|---|---|---|---|---|
| No. | benzodioxane | G₄ | G₅ | E | NMR | MS |
| 10 | 6 | 4′-CH₃ | H | H | (DMSO-d6): 2.26 | a = 346.1 |
| | | | | | (3H, s); 4.16 (4 | b = 368.1 |
| | | | | | H, m); 6.72 (1H, | c = 344.1 |
| | | | | | m); 6.88 (1H, m); | |
| | | | | | 7.04-7.21 (3H, | |
| | | | | | m); 7.24-7.33 (2 | |
| | | | | | H, m); 7.34-7.60 | |
| | | | | | (4H, m); 10.01 (1 | |
| | | | | | H, broad s). | |
| 11 | 6 | 4′-CH₂CH₃ | H | H | (DMSO-d6): 1.16 | a = 360.1 |
| | | | | | (3H, t, J = 9 Hz); | b = 382.1 |
| | | | | | 2.57 (2H, q, J = | c = 358.1 |
| | | | | | 9 Hz); 4.17 (4H, | |
| | | | | | m); 6.73 (1H, m); | |
| | | | | | 6.89 (1H, m); | |
| | | | | | 7.11 (1H, m); | |
| | | | | | 7.16-7.25 (2H, | |
| | | | | | m); 7.28-7.37 (2 | |
| | | | | | H, m); 7.37-7.58 | |
| | | | | | (4H, m); 10.03 (1 | |
| | | | | | H, broad s). | |
| 12 | 6 | H | H | H | — | a = 332.1 |
| | | | | | | b = 354.1 |
| | | | | | | c = 330.1 |
| 13 | 6 | 4′-CH(CH₃)₂ | H | H | — | a =374.1 |
| | | | | | | b = 396.1 |
| | | | | | | c = 372.1 |
| 14 | 6 | 4′-OCF₃ | H | H | — | a = 416.1 |
| | | | | | | b = 438.1 |
| | | | | | | c = 414.1 |
| 15 | 6 | 3′-CF₃ | H | H | — | a = 400.1 |
| | | | | | | b = 422.1 |
| | | | | | | c = 398.1 |
| | | | | | | d = 444.1 |
| 16 | 7 | 4′-CH(CH₃)₂ | 2-CO—OEt | H | — | a = 446.2 |
| 17 | 7 | 4′-CH(CH₃)₂ | 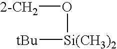 | H | — | a = 518.3 |
| 18 | 7 | 4′-CH(CH₃)₂ | 2-CH₂—OH | H | — | a = 404.3 |
| 19 | 7 | 4′-CH(CH₃)₂ | 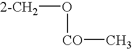 | H | — | a = 446.3 |
| 20 | 7 | 4′-CH(CH₃)₂ | 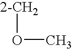 | H | — | a = 418.1 |
| 21 | 7 | 4′-CH(CH₃)₂ | 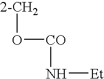 | H | — | a = 475.5 c = 473.3 |
| 22 | 7 | 4′-CH(CH₃)₂ | 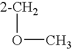 | CH₃ | — | a = 432.4 |
| 23 | 7 | 4′-OCF₃ | 2-CH₂—OH | H | — | a = 446.1 |
| | | | | | | c = 444.1 |
| 24 | 7 | 4′-OCF₃ | 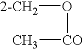 | H | — | a = 488.2 c = 486.1 |
| 25 | 7 | 4′-OCF₃ | 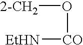 | H | — | a = 517.4 |
| 26 | 7 | 4′-OCF₃ | 2-CH₂OCH₃ | H | — | a = 460.1 |
| | | | | | | c = 458.0 |
| 27 | 7 | 4′-CH(CH₃)₂ | 2-CH₂—OH | CH3 | — | a = 418.3 |
| 28 | 7 | 4′-OCF₃ | 2-CH₂—OH | CH3 | — | a = 460.4 |
| 29 | 7 | 4′-CH(CH₃)₂ | 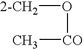 | CH3 | — | a = 480.4 |

a corresponds to ES+M+H
b corresponds to ES+M+23
c corresponds to ES−M−H

d corresponds to ES−M+HCOO⁻, this definition is also valid hereinbelow.

TABLE 4

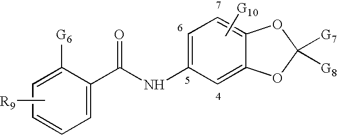

| Example | | | | | | | |
|---|---|---|---|---|---|---|---|
| No. | G₆ | G₇ | G₈ | G₉ | G₁₀ | NMR | MS |
| 30 | 4′-CF₃-phenyl | F | F | H | H | (DMSO-d6): 7.20 | a = 422 |
| | | | | | | (1H, m); 7.33 (1 | b = 444 |
| | | | | | | H, m); 7.50-7.73 | c = 420 |
| | | | | | | (7H, m); 7.76 (2 | |
| | | | | | | H, m); 10.58 (1H, | |
| | | | | | | broad s). | |
| 31 | 4′-methylphenyl | F | F | H | H | (DMSO-d6): 2.26 | a = 368.1 |
| | | | | | | (3H, s); 7.13-7.25 | b = 390.1 |
| | | | | | | (3H, m); 7.25- | c = 366.1 |
| | | | | | | 7.38 (3H, m); | |
| | | | | | | 7.38-7.70 (5H, | |
| | | | | | | m); 10.43 (1H, | |
| | | | | | | broad s). | |
| 32 | 4′-ethylphenyl | F | F | H | H | (DMSO-d6): 1.15 | a = 382.1 |
| | | | | | | (3H, t, J = 9 Hz); | b = 404.1 |
| | | | | | | 2.58 (2H, q, J = 9 | c = 380.1 |
| | | | | | | Hz); 7.15-7.26 (3 | |
| | | | | | | H, m); 7.27-7.37 | |
| | | | | | | (3H, m); 7.41- | |
| | | | | | | 7.51 (2H, m); 7.65 | |
| | | | | | | (1H, m); 10.44 (1 | |
| | | | | | | H, broad s). | |
| 33 | 4′-CH(CH₃)₂phenyl | F | F | H | H | (DMSO-d6): 1.17 | a = 396.1 |
| | | | | | | (6H, d, J = 6 Hz); | b = 418.1 |
| | | | | | | 2.86 (1H, sept, J | c = 394.1 |
| | | | | | | = 6 Hz); 7.07-7.40 | |
| | | | | | | (6H, m); 7.42- | |
| | | | | | | 7.71 (5H .m); | |
| | | | | | | 10.41 (1H, broad s). | |
| 34 | 3′-methyl-4′-methyl- | F | F | H | H | (DMSO-d6): 2.11- | a = 380.3 |
| | phenyl | | | | | 2.21 (6H, 2 s); | |
| | | | | | | 7.05-7.17 (2H, | |
| | | | | | | m); 7.17-7.25 (2 | |
| | | | | | | H, m); 7.26-7.36 | |
| | | | | | | (1H, m); 7.38- | |
| | | | | | | 7.48 (2H, m); | |
| | | | | | | 7.48-7.58 (2H, | |
| | | | | | | m); 7.60-7.65 (1 | |
| | | | | | | H, m); 10.43 (1H, | |
| | | | | | | broad s). | |
| 35 | 4′-chlorophenyl | F | F | H | H | (DMSO-d6): 7.20 | a = 388 |
| | | | | | | (1H, m); 7.32 (1 | b = 410 |
| | | | | | | H, m); 7.37-7.55 | c = 386 |
| | | | | | | (6H, m); 7.55- | |
| | | | | | | 7.64 (2H, m); 7.68 | |
| | | | | | | (1H, m); 10.51 (1 | |
| | | | | | | H, broad s). | |
| 36 | 4′-methoxyphenyl | F | F | H | H | (DMSO-d6): 3.72 | a = 384 |
| | | | | | | (3H, s); 6.93 (2 H, | b = 406 |
| | | | | | | m); 7.20 (1H, m); | c = 382 |
| | | | | | | 7.25-7.38 (3H, | |
| | | | | | | m); 7.38-7.48 (2 | |
| | | | | | | H, m); 7.49-7.59 | |
| | | | | | | (2H, m) 7.66 (1H, | |
| | | | | | | m); 10.42 (1H, | |
| | | | | | | broad s). | |
| 37 | N-pyrrolyl | F | F | H | H | — | a = 343 |
| | | | | | | | b = 365 |
| | | | | | | | c = 341 |
| 38 | phenyl | F | F | H | H | — | a = 354 |
| | | | | | | | b = 376 |
| | | | | | | | c = 352 |
| 39 | 3′-CF₃-phenyl | F | F | H | H | — | a = 423 |
| | | | | | | | c = 419.9 |
| 40 | 4′-OCF₃-phenyl | F | F | H | H | — | a = 438 |
| | | | | | | | b = 460 |
| | | | | | | | c = 437 |
| 41 | 4′-CH(CH₃)₂ phenyl | H | H | H | H | — | a = 360.3 |
| 42 | 4′-ethylphenyl | F | F | 3-CH₃ | H | — | a = 396.3 |
| | | | | | | | c = 394.2 |
| 43 | 4′-methoxymethyl- | F | F | H | H | — | a = 398.2 |
| | phenyl | | | | | | c = 396.2 |
| 44 | 4′-(1-methoxyethyl)- | F | F | H | H | — | a = 412.2 |
| | phenyl | | | | | | c = 410.3 |
| 45 | 4′-tBu-phenyl | F | F | H | H | — | a = 410.4 |
| | | | | | | c = 408.3 | |
| 46 | 4′-methylcarbonyl- | F | F | H | H | — | a = 426.4 |
| | oxymethylphenyl | | | | | | c = 424.2 |
| 47 | 4′-isopropyloxy | F | F | H | H | — | a = 412.3 |
| | phenyl | | | | | | c = 410.2 |
| 48 | 4′-ethylaminocarb- | F | F | H | H | — | a = 469.5 |
| | oxyloxyethy-phenyl | | | | | | c = 467.3 |
| 49 | 4′-trifluoromethoxy- | F | F | 3-CH₃ | H | — | a = 452.4 |
| | phenyl | | | | | | c = 450.2 |
| 50 | 4′-methoxycarbonyl- | F | F | H | H | — | a = 440.5 |
| | ethylphenyl | | | | | | c = 438.8 |
| 51 | 4′-trifluoromethoxy- | H | H | H | H | — | a = 402.3 |
| | phenyl | | | | | | c = 400.2 |
| 52 | 4′-methylcarbonyl- | F | F | H | H | — | a = 440.4 |
| | oxyethylphenyl | | | | | | c = 438.4 |
| 53 | 4′-(2-hydroxyethyl)- | F | F | H | H | — | a = 398.4 |
| | phenyl | | | | | | c = 396.3 |
| 54 | 4′-isopropylcarbon- | F | F | H | H | — | a = 424.2 |
| | ylphenyl | | | | | | c = 422.1 |
| 55 | 4′-(2-hydroxy-3- | F | F | H | H | — | a = 426.3 |
| | methylpropyl)phenyl | | | | | | c = 424.3 |
| 56 | 4′-trifluoromethoxy- | H | H | 3-CH₃ | 7- | — | a = 446.1 |
| | phenyl | | | | OCH₃ | | c = 444.2 |
| 57 | 4′-(2-methylpropyl)- | F | F | H | H | — | a = 410.2 |
| | phenyl | | | | | | c = 408.2 |
| 58 | 4′-isopropylphenyl | H | H | H | 7- | — | a = 390.2 |
| | | | | | OCH₃ | | c = 388.1 |
| 59 | 4′-isopropylphenyl | H | H | H | 7-iso- | — | a = 418.3 |
| | | | | | propoxy | | c = 416.3 |
| 60 | 4′-trifluoromethoxy- | H | H | 3- | 7-iso- | — | a = 474.4 |
| | phenyl | | | methyl | propoxy | | c = 472.3 |

TABLE 5

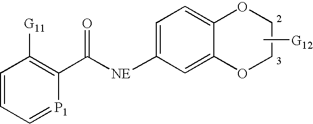

| Example | | | | | | |
|---|---|---|---|---|---|---|
| No. | G₁₁ | P₁ | G₁₂ | E | NMR | MS |
| 61 | N-pyrrolyl | C | H | H | — | a = 321.1 |
| | | | | | | b = 343.1 |
| | | | | | | c = 319.1 |
| 62 | phthalimido | C | H | H | — | a = 401.1 |
| | | | | | | b = 423.1 |
| | | | | | | c = 399.1 |
| 63 | phenyl | N | H | H | — | a = 333.1 |
| | | | | | | b = 355.1 |
| | | | | | | c = 331.1 |
| 64 | 4′-(trifluoromethoxy)- | C | 3-CH₂OH | CH₃ | — | a = 460.4 |
| | phenyl | | | | | |
| 65 | 4′-isopropylphenyl | C | 3-CH₂OCOCH₃ | CH₃ | — | a = 460.4 |

TABLE 6

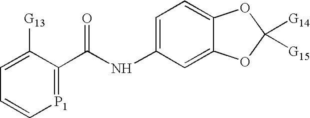

| Example | | | | | | |
|---|---|---|---|---|---|---|
| No. | G₁₃ | P₂ | G₁₄ | G₁₅ | NMR | MS |
| 66 | phenyl | N | F | F | — | a = 355 |
| | | | | | | b = 377 |
| | | | | | | c = 353 |
| 67 | 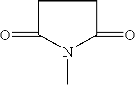 | C | F | F | — | a = 375 c = 373 |
| 68 | N-imidazolylethyl- | C | F | F | — | a = 448.4 |
| | phenyl | | | | | c = 446.2 |
| 69 | 4-(2-methyl-1-prop- | C | F | F | — | a = 408.2 |
| | enyl)phenyl | | | | | c = 406.2 |

TABLE 7

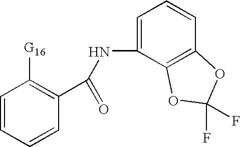

| | | | |
|---|---|---|---|
| Example | | | |
| No. | G₁₆ | NMR | MS |

| | | | |
|---|---|---|---|
| 70 | 4′-ethylphenyl | — | a = 382.3 |
| | | | c = 380.2 |
| 71 | 4′-isopropylphenyl | — | a = 396.3 |
| | | | c = 394.3 |
| 72 | 4′-trifluoromethoxyphenyl | — | a = 438.2 |
| | | | c = 436.2 |

TABLE 8

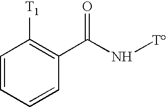

| Example | | | | |
|---|---|---|---|---|
| No. | T¹ | T* | NMR | MS |
| 73 | 4′-ethylphenyl | 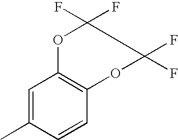 | — | a = 432.3 c = 430.2 |
| 74 | 4′-isopropylphenyl | 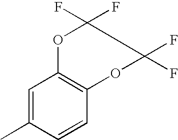 | — | a = 446.3 c = 444.3 |
| 75 | 4′-trifluoromethoxyphenyl | 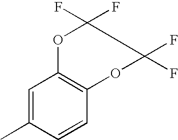 | — | a = 488.2 c = 486.2 |
| 76 | 4′-ethylphenyl | 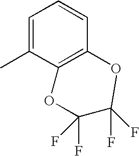 | — | a = 432.3 c = 430.3 |
| 77 | 4′-isopropylphenyl | 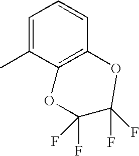 | — | a = 446.3 c = 444.3 |
| 78 | 4′-trifluoromethoxyphenyl | 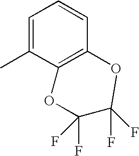 | — | a = 488.3 c = 486.3 |
| 79 | 4′-ethylphenyl | 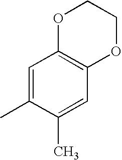 | — | a = 374.3 |
| 80 | 4′-isopropylphenyl | 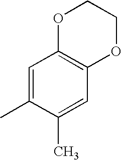 | — | a = 388.3 |
| 81 | 4′-trifluoromethoxyphenyl | 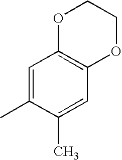 | — | a = 430.2 c = 428.2 |

### Example 82

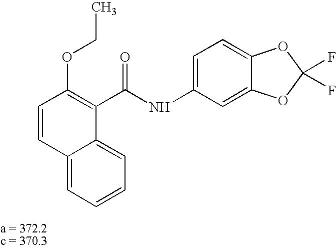

### Examples 83 to 106

The invention is further illustrated by Examples 83 to 106 below defined in Table 9 below, the characterisation data for which are collated in Table 10 below.

| Ex. | T | A | R | B | 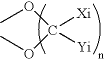 |
|---|---|---|---|---|---|
| 83 | 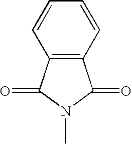 | 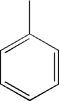 | H | 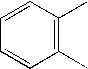 | 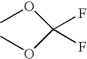 |
| 84 | 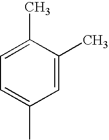 | 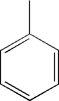 | H | 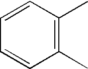 | 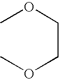 |
| 85 | 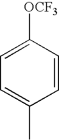 | 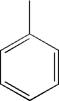 | H | 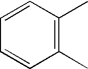 | 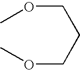 |
| 86 | 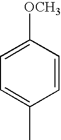 | 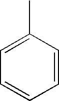 | H | 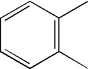 | 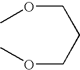 |
| 87 | 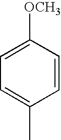 | 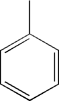 | H | 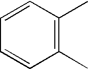 | 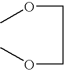 |
| 88 | 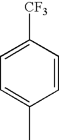 | 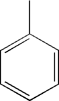 | H | 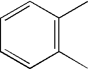 | 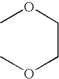 |
| 89 | 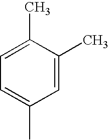 | 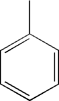 | H | 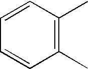 | 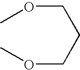 |
| 90 | 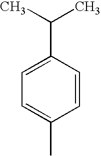 | 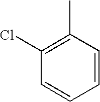 | H | 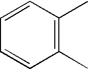 | 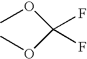 |
| 91 | 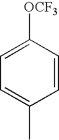 | 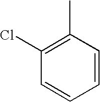 | H | 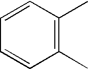 | 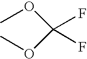 |
| 92 | 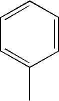 | 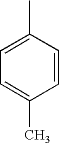 | H | 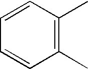 | 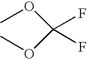 |
| 93 | 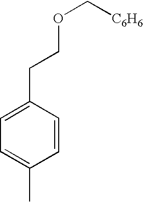 | 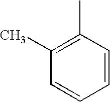 | H | 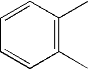 | 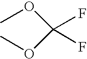 |
| 94 | 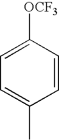 | 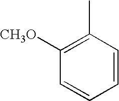 | H | 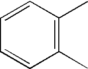 | 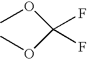 |
| 95 | 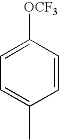 | 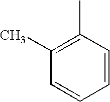 | H | 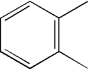 | 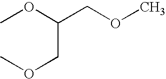 |
| 96 | 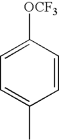 | 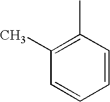 | H | 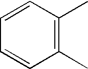 | 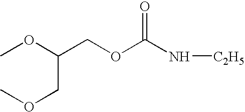 |
| 97 | 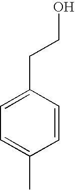 | 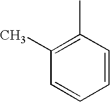 | H | 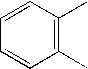 | 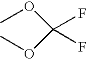 |
| 98 | 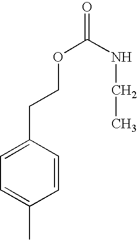 | 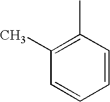 | H | 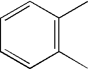 | 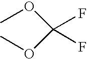 |
| 99 | 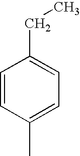 | 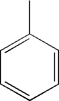 | H | 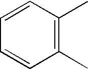 | 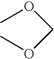 |
| 100 | 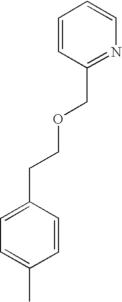 | 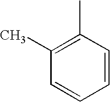 | H | 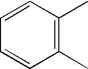 | 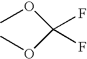 |
| 101 | 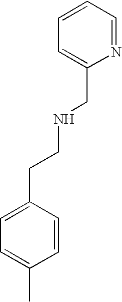 | 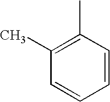 | H | 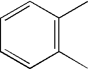 | 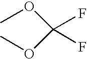 |
| 102 | 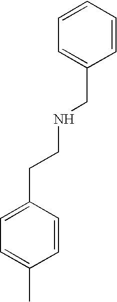 | 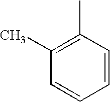 | H | 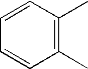 | 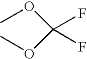 |
| 103 | 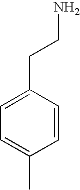 | 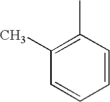 | H | 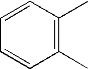 | 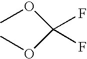 |
| 104 | 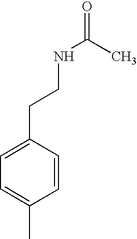 | 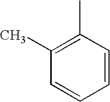 | H | 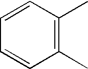 | 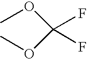 |
| 105 | 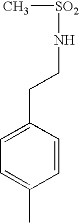 | 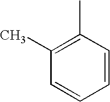 | H | 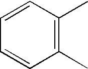 | 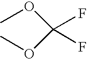 |
| 106 | 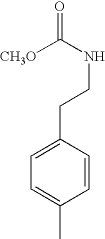 | 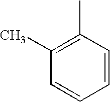 | H | 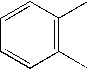 | 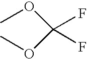 |

TABLE 10

| Example | Spectral data (MS) |
|---|---|
| 83 | a = 423 |
| | c = 421 |
| 84 | a = 360.1 |
| 85 | a = 430.1 |
| | c = 428.1 |
| | d = 474.1 |
| 86 | a = 376.3 |
| 87 | a = 362.2 |
| 88 | a = 400.3 |
| | c = 398.2 |
| | d = 444.2 |
| 89 | a = 374.3 |
| | c = 372.3 |

### Example 93

### a) Preparation of 2-bromo-3-methylbenzoyl chloride

6.0 g (27.9 mmol, 1.0 eq) of 2-bromo-3-methylbenzoic acid are dissolved in 140 ml of dichloromethane, and 7.3 g, 5.0 ml (83.7 mmol; 3.0 eq) of oxalyl chloride are then added to the reaction medium maintained at 0° C. The solution is then heated to 50° C. and maintained at this temperature for four hours, after which the reaction medium is concentrated under reduced pressure for one hour. The infrared spectrum of the product obtained shows a peak revealing the carbonyl function of the acid chloride at 1777 cm⁻¹. The product is obtained in quantitative yield and is used without further modification (6.5 g; 100%).

### b) Coupling of 2-bromo-3-methylbenzoyl chloride and 5-amino-2,2-difluorobenzo-1,3-dioxole

2.0 g (11.7 mmol; 1.0 eq) of 5-amino-2,2-difluorobenzo-1,3-dioxole are dissolved in 100 ml of acetonitrile, and 2.5 ml (17.6 mmol; 1.5 eq) of triethylamine and a catalytic amount of 4-dimethylaminopyridine are then added to the reaction medium. The solution is then cooled to 0° C. and 3.0 g (12.9 mmol; 1.1 eq) of 2-bromo-3-methylbenzoyl chloride in 40 ml of acetonitrile are added dropwise to this solution. The reaction medium is stirred overnight and then concentrated under reduced pressure. The residue is then dissolved in dichloromethane and washed with aqueous 10% potassium carbonate solution, with water, with aqueous 10% citric acid solution and with saline solution. The organic phase is then dried over anhydrous magnesium sulfate and concentrated under reduced pressure. The solid obtained is purified by flash chromatography on silica, using dichloromethane as eluent. A cream-coloured solid is thus obtained (2.35 g; 54%).

### c) Preparation of benzyl 2-(4-bromophenyl)ethyl ether

4.1 g (102 mmol; 2.2 eq) of a 60% dispersion of sodium hydride in mineral oil are washed twice with diethyl ether and then treated with 6.6 ml (55.6 mmol; 1.2 eq) of benzyl bromide at 0° C. 9.3 g (46.3 mmol; 1.0 eq) of 2-(4-bromophenyl) ethanol are added dropwise to a solution of 250 ml of N,N-dimethylformamide at 0° C., after which the mixture is allowed to warm to room temperature overnight. The next day, the solution is diluted with ethyl acetate and then mixed cautiously with water, after which it is washed with 2.0 M sodium hydroxide solution and then with saline solution. The organic phase is dried over anhydrous magnesium sulfate and concentrated under reduced pressure. The resulting oil is then purified by flash chromatography on silica, first using hexane as eluent so as to elute all the residual benzyl bromide, and then using a mixture of ethyl acetate and hexane in a 1:6 ratio so as to elute the expected product in the form of a colourless oil (12.67 g; 94%).

### d) Preparation of Pinacol Boronate from the Bromide Obtained in Step c)

0.216 g (0.31 mmol; 3.0 mol %) of bis(triphenylphosphine)palladium (II) chloride is dissolved in 40 ml of dioxane, 3.0 g (10.3 mmol; 1.0 eq) of the ether prepared in step c) above are then added to this solution and the reaction mixture is stirred for 10 minutes, followed by addition of 4.4 ml of triethylamine to the reaction medium. After reaction for 10 minutes, 2.25 ml (15.5 mmol; 1.5 eq) of pinacol borane are added to the reaction medium. The solution is heated at 100° C. for four hours and then cooled to room temperature. The reaction medium is diluted by adding ethyl acetate and then mixed cautiously with water. The organic phase is separated out, washed with water and with saline solution and then dried over anhydrous magnesium sulfate and concentrated under reduced pressure. The resulting oil is then purified by flash chromatography on silica, using a mixture of dichloromethane/hexane in 2:1 proportions as eluent. The product obtained is a colourless oil (2.05 g; 59%).

### e) Preparation of the Corresponding Boronic Acid of the Formula

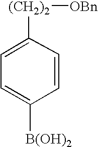

1.0 g (3.0 mmol; 1.0 eq) of pinacol boronate is dissolved in 150 ml of acetone, after which 2.3 g (10.7 mmol; 3.6 eq) of sodium periodate, 0.83 g (10.7 mmol; 3.6 eq) of ammonium acetate and 70 ml of water are added to the reaction medium. The solution is stirred for 48 hours at room temperature and the acetone is then removed under reduced pressure. Aqueous 2.0 M sodium hydroxide solution (150 ml) is then added and the solution is mixed for one hour and then extracted by adding dichloromethane. The aqueous phase is cooled to 0° C. and then acidified cautiously using concentrated HCl, to pH=3. The aqueous phase is then extracted twice with ethyl acetate, after which it is dried over anhydrous magnesium sulfate and concentrated under reduced pressure so as to give the expected boronic acid in the form of a colourless oil (0.42 g; 55%). This compound is used in the next reaction without further modification.

### f) Compound of Example 93

0.122 g (0.33 mmol; 1.0 eq) of the bromide prepared in step b) and 0.100 g (0.39 mmol; 1.2 eq) of the boronic acid prepared in step e) are dissolved in 1.0 ml of acetonitrile and aqueous 0.4 M sodium carbonate solution (1.0 ml) is then added, followed by addition of 0.011 g (0.01 mmol; 3 mol %) of Pd(PPh₃)₄. The reaction medium is refluxed at 83° C. overnight and, after cooling, it is then diluted with water and extracted with ethyl acetate. The organic phase is washed with water and then with solution saline, after which it is dried over anhydrous magnesium sulfate and then concentrated under reduced pressure. The residue is purified by flash chromatography on silica, using a mixture of ethyl acetate/hexane in a 1:5 ratio, so as to give the expected coupling product in the form of a colourless oil (0.082 g; 49%).

As a variant, the compound of Example 93 can be prepared by carrying out steps a′) to e′) below.

### a′) Preparation of methyl 2-bromo-3-methylbenzoate

25.0 g (11.6 mmol; 1.0 eq) of 2-bromo-3-methylbenzoic acid and 22.0 g (11.6 mmol) of para-toluenesulfonic acid are dissolved in 580 ml of methanol and refluxed overnight. After cooling, the reaction medium is concentrated under reduced pressure. The residue is dissolved in ether and then washed twice with saturated aqueous sodium hydrogen carbonate solution and then with water and with solution saline. The organic phase is then dried over anhydrous magnesium sulfate and concentrated under reduced pressure. The expected product is obtained in the form of a pale yellow oil (23.6 g; 89%) and is then used without further purification.

### b′) Coupling of the Methyl Ester Obtained in Step a′) with the Boronic Acid Prepared in Step e) Above

Aqueous 0.4 M sodium carbonate solution (1.0 ml) is added to 0.075 g (0.33 mmol; 1.0 eq) of the bromide prepared in step a′) above and 0.100 g (0.39 mmol; 1.2 eq) of the boronic acid prepared in step e) above and dissolved in 1.0 ml of acetonitrile, followed by addition of 0.011 g (0.01 mmol; 3 mol %) of Pd(PPh₃)₄. The reaction medium is refluxed at 83° C. overnight. After cooling, the reaction medium is diluted with water and then extracted with diethyl ether. The organic phases are washed with water and then with saline solution, after which the resulting solution is dried over anhydrous magnesium sulfate and concentrated under reduced pressure. The expected product is purified by flash chromatography using a mixture of ethyl acetate and hexane in a 1:12 ratio, so as to give the expected product in the form of a colourless oil (0.079 g; 66%).

### c′) Saponification of the Ester

0.073 g (0.203 mmol; 1.0 eq) of the methyl ester obtained in step b′) above is dissolved in 1.0 ml of methanol, followed by addition of aqueous 2.0 M sodium hydroxide solution (0.35 ml; 0.709 mmol; 3.5 eq) to the reaction medium with stirring. The reaction medium is then heated at 60° C. overnight, after which it is concentrated under reduced pressure. The residue is dissolved in 20 ml of water and then acidified with 2.0 M hydrochloric acid solution until a precipitate is obtained, which is extracted with ethyl acetate. The organic phases are washed with water and then dried over anhydrous magnesium sulfate and concentrated under reduced pressure. The resulting white solid is dried under vacuum and used in crude form (0.06 g; 86%).

### d′) Synthesis of the Corresponding Acid Chloride

0.06 g (0.17 mmol; 1.0 eq) of the carboxylic acid obtained in step c′) above is dissolved in 0.085 ml of dichloromethane, and 0.032 ml of oxalyl chloride is then added at 0° C. The solution is heated at 50° C. for four hours and then concentrated under reduced pressure for one hour. The infrared spectrum of the product reveals the presence of the carbonyl function of the acid chloride at 1777 cm⁻¹. The expected product is obtained in quantitative yield, and is used in crude form in the next step (0.062 g; 100%).

### e′) Coupling of the acid chloride from step d′) with 5-amino-2,2-difluorobenzo-1,3-dioxole

0.028 g (0.16 mmol; 1.0 eq) of 5-amino-2,2-difluorobenzo-1,3-dioxole is dissolved in 1.0 ml of acetonitrile, followed by addition of 0.034 ml (0.26 mmol; 1.6 eq) of triethylamine and a catalytic amount of 4-dimethylaminopyridine. The solution is cooled to 0° C., after which 0.062 g (0.17 mmol; 1.1 eq) of the acid chloride obtained in step d′) above in 1.0 ml of acetonitrile is added dropwise to the reaction medium. The reaction medium is then stirred overnight, after which it is concentrated under reduced pressure. The residue is dissolved in dichloromethane and then washed with aqueous 10% potassium carbonate solution, with water, with aqueous 10% citric acid solution, with water and then with saline solution. The organic phase is then dried over anhydrous magnesium sulfate and concentrated under reduced pressure. The solid obtained is purified by flash chromatography on silica, using as eluent a mixture of ethyl acetate and hexane in a 1:4 ratio, so as to obtain the expected product in the form of a colourless oil (0.072 g; 90%).

1- Compound of the formula I: 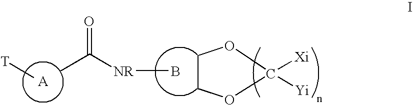
in which
A and B independently represent an optionally substituted phenyl nucleus; or an optionally substituted pyridyl nucleus;
T represents an optionally substituted, saturated and/or unsaturated aromatic carbocyclic nucleus; an optionally substituted, saturated and/or unsaturated aromatic heterocyclic nucleus; or
T represents a saturated and/or unsaturated aromatic carbocyclic nucleus which is fused to the nucleus A, is optionally substituted and is linked to two adjacent carbon atoms belonging to the nucleus A;
R represents a hydrogen atom; an optionally substituted saturated aliphatic hydrocarbon-based group; or an optionally substituted, saturated or unsaturated aromatic carbocyclic group;
n represents an integer chosen between 1, 2, 3, 4 and 5;
the radicals Xᵢ and Yᵢ are independently chosen from a hydrogen atom; a halogen atom; an optionally substituted, saturated and/or unsaturated aliphatic hydrocarbon-based group; an optionally substituted, saturated or unsaturated aromatic carbocyclic nucleus; a-u¹-COOL group, in which u¹ represents a bond or an alkylene group and L is an optionally substituted saturated aliphatic hydrocarbon-based group or an optionally substituted, saturated and/or unsaturated aromatic carbocyclic group; -u²-SiR¹R²R³, in which u² represents a bond, an alkylene group or an alkyleneoxy group in which the oxygen atom is linked to Si and R¹, R² and R³ independently represent an optionally substituted saturated aliphatic hydrocarbon based group; -u³-OW, in which u³ represents a bond or an alkylene group and W may represent a hydrogen atom or is as defined above for L; u⁴-CO-G, in which u⁴ represents a bond, an alkylene group or an alkyleneoxy group in which the oxygen atom is linked to the carbonyl group and G is as defined above for L; -u⁵-CO—NH-J, in which u⁵ represents a bond, an alkylene group or an alkyleneoxy group in which the oxygen atom is linked to the carbonyl group and J is as defined above for L; or a radical Xi and a radical Yi both attached to the same carbon atom, together with this carbon atom, represent an optionally substituted saturated carbocyclic nucleus;
and the pharmaceutically usable derivatives, solvates and stereoisomers thereof comprising a mixture thereof in all proportions.

2- Compound according to claim 1, in which A and B represent optionally substituted phenyl.

3- Compound according to claim 1, in which B represents optionally substituted phenyl; and A represents optionally substituted pyridyl.

4- Compound according to claim 1, characterised in that T represents an optionally substituted monocyclic or bicyclic aryl nucleus; a saturated or unsaturated, monocyclic or bicyclic aromatic heterocyclic nucleus containing 1 to 3 heteroatoms chosen from N, O and S, the said nucleus being optionally substituted by one or more radicals chosen from oxo, a halogen atom, alkyl which is optionally halogenated and/or optionally interrupted by one or more oxygen or sulfur atoms; -alk¹-O—CO—R⁴, in which alk¹ is an alkylene radical and R⁴ represents alkyl or alkylamino; -alk²-CO—O—R⁵, in which alk² is an alkylene radical and R⁵ is as defined above for R⁴; —CO—R⁶, in which R⁶ is as defined above for R⁴; hydroxyalkyl; -alk³-TT-Q, in which alk³ represents alkylene, TT represents O or NH, and Q represents an optionally substituted arylalkyl nucleus; optionally substituted heteroarylalkyl; —CO—K, in which K represents alkyl or alkoxy; or —SO₂—K in which K is as defined above; -alk⁴-O—CO—NH-alk⁵, in which -alk⁴ and alk⁵ independently represent alkylene; aminoalkyl; hydroxyalkyl, heteroarylalkyl, preferably imidazolylalkyl; and alkenyl.

5- Compound according to claim 1, characterised in that R is chosen from H and alkyl.

6- Compound according to claim 1, characterised in that n represents 1, 2 or 3.

7- Compound according to claim 1, characterised in that the radicals Xi and Yi are independently chosen from a hydrogen atom; a halogen atom; an alkyl group which is optionally interrupted by one or more oxygen or sulfur atoms; a hydroxyalkyl group; —COOL, in which L is as defined in claim 1; -alk³-SiR¹R²R³, in which alk³ represents alkylene and R¹, R² and R³ are as defined in claim 1; -alk⁴-O—CO-alk⁵, in which alk⁴ and alk⁵ independently represent alkyl; -alk⁶-O—CO—NH-alk⁷, in which alk⁶ and alk⁷ independently represent alkyl.

8- Compound according to claim 1, characterised in that A represents phenyl which is optionally substituted by halogen, alkyl or alkoxy, and in that B represents phenyl which is optionally substituted by halogen, alkyl or alkoxy.

9- Compound according to claim 1, characterised in that A represents pyridyl; B represents phenyl; n represents 1, 2 or 3; R represents H; and the radicals Xi and Yi represent a hydrogen atom or a fluorine atom.

10- Compound according to claim 1, characterised in that the radicals Xi and Yi attached to the same carbon atom are identical and both represent a hydrogen atom or both represent a fluorine atom.

11- Compound according to claim 1, characterised in that T represents a nucleus chosen from phenyl, pyrrolyl, phthalimidyl and succinimidyl, the said nucleus being optionally substituted by one or more radicals chosen from:
alkyl which is optionally halogenated and/or optionally interrupted by one or more oxygen or sulfur atoms;
alk¹-O—CO—R⁴, in which alk¹ is an alkylene radical and R⁴ represents alkyl or alkylamino;
alk²-CO—O—R⁵, in which alk² is an alkylene radical and R⁵ is as defined above for R⁴;
CO—R⁶, in which R⁶ is as defined above for R⁴;
hydroxyalkyl;
heteroarylalkyl, preferably imidazolylalkyl; and
alkenyl.

12- Compound of the formula I according to claim 1, chosen from:
5-(4′-trifluoromethylbiphen-2-ylcarbonylamino)-2,2-difluorobenzo[1,3]dioxole;
5-(4′-isopropylbiphen-2-ylcarbonylamino)-2,2-difluorobenzo[1,3]dioxole;
5-(4′-methoxybiphen-2-ylcarbonylamino)-2,2-difluorobenzo[1,3]dioxole;
5-(4′-trifluoromethoxybiphen-2-ylcarbonylamino)-2,2-difluorobenzo[1,3]dioxole;
5-(4′-isopropylbiphen-2-ylcarbonylamino)benzo[1,3]dioxole;
5-(4′-ethyl-3-methylbiphen-2-ylcarbonylamino)-2,2-difluorobenzo[1,3]dioxole;
5-(4′-ethylaminocarbonyloxyethylbiphen-2-ylcarbonylamino)-2,2-difluorobenzo[1,3]dioxole;
5-(4′-trifluoromethoxy-3-methylbiphen-2-ylcarbonylamino)-2,2-difluorobenzo[1,3]dioxole;
5-(4′-methoxycarbonylethylbiphen-2-ylcarbonylamino)-2,2-difluorobenzo [1,3]dioxole;
4′-isopropylbiphenyl-2-carboxylic acid (3-methoxymethyl-2,3-dihydrobenzo[1,4]dioxin-6-yl)amide;
7-[(4′-isopropylbiphenyl-2-carbonyl)amino]-2,3-dihydrobenzo[1,4]dioxin-2-ylmethyl ethylcarbamate;
4′-ethylbiphenyl-2-carboxylic acid (2,2-difluorobenzo[1,3]dioxol-5-yl)amide;
4′-trifluoromethoxybiphenyl-2-carboxylic acid benzo[1,3]dioxol-5-ylamide;
4′-(2-hydroxyethyl)biphenyl-2-carboxylic acid (2,2-difluorobenzo[1,3]dioxol-5-yl)amide;
4′-isobutylbiphenyl-2-carboxylic acid (2,2-difluorobenzo[1,3]dioxol-5-yl)amide;
4′-(2-methylpropenyl)biphenyl-2-carboxylic acid (2,2-difluorobenzo[1,3]dioxol-5-yl)amide;
6-chloro-4′-isopropylbiphenyl-2-carboxylic acid (2,2-difluorobenzo[1,3]dioxol-5-yl)amide;
6-chloro-4′-trifluoromethoxybiphenyl-2-carboxylic acid (2,2-difluorobenzo[1,3]dioxol-5-yl)amide;
4′-(2-benzyloxyethyl)-6-methylbiphenyl-2-carboxylic acid (2,2-difluorobenzo[1,3]dioxol-5-yl)amide;
6-methoxy-4′-trifluoromethoxybiphenyl-2-carboxylic acid (2,2-difluorobenzo[1,3]dioxol-5-yl)amide;
6-methyl-4′-trifluoromethoxybiphenyl-2-carboxylic acid (2-methoxymethyl-2,3-dihydrobenzo[1,4]dioxin-6-yl)amide;
6-[(6-methyl-4′-trifluoromethoxybiphenyl-2-carbonyl)amino]-2,3-dihydrobenzo [1,4]dioxin-2-ylmethyl ethylcarbamate;
2-[6′-(2,2-difluorobenzo[1,3]dioxol-5-ylcarbamoyl)-2′-methylbiphenyl-4-yl]ethyl ethylcarbamate;
4′-ethylbiphenyl-2-carboxylic acid benzo[1,3]dioxol-5-ylamide.

13- Process for the preparation of compounds of the formula I as defined in claim 1, characterised in that a carboxylic acid of the formula II: 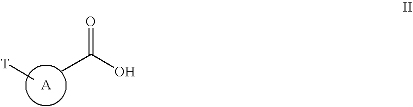
in which A and T are as defined in claim 1, optionally in activated form, is reacted with an amine of the formula III: 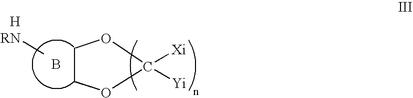
in which R, Xi, Yi, n and B are as defined in claim 1.

14- Process for the preparation of compounds of the formula I in which R represents an optionally substituted saturated aliphatic hydrocarbon-based group; or an optionally substituted, saturated or unsaturated aromatic carbocyclic group, the said process comprising the reaction of the amino function attached to the nuclei A and B of the corresponding compound of the formula I in which R 5- represents a hydrogen atom, with a suitable electrophilic site.
Compound of the formula XXI a 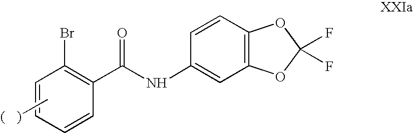
in which ( [symbol] denotes the possible substituent(s) on the phenyl group to which ( ) is attached, which are chosen from halogen, alkyl and alkoxy.
Compound according to claim 15, for which ( ) denotes methyl.
Compound of the formula XIVa: 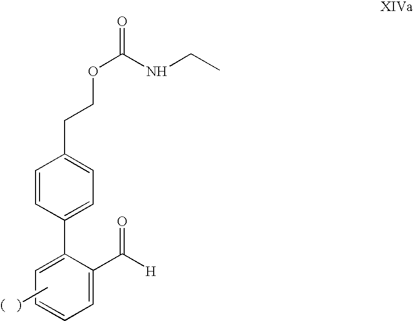
in which ( [symbol] denotes the possible substituent(s) on the phenyl group to which ( ) is attached, which are chosen from halogen, alkyl and alkoxy.

18- Compound according to claim 17 of the formula XIVa, for which ( ) denotes a hydrogen atom or a methyl group.

19- Compound of the formula IIb: 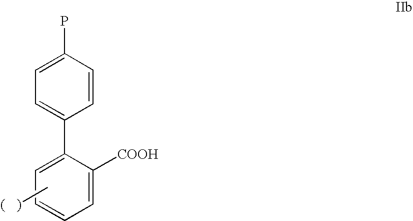
in which
P is chosen from —OCF₃ provided that ( ) does not represent hydrogen;
—CO—CH(CH₃)₂; —(CH₂)₂—O—CO—CH₃; —(CH₂)₂—CO—O—CH₃; and —CH₂)₂—O—CO—NH—CH₂—CH₃;
( [symbol] denotes the possible substituent(s) on the phenyl group to which ( ) is attached, which are chosen from hydrogen, halogen, such as chlorine, alkyl, such as methyl and alkoxy, such as methoxy.

20- Compound according to claim 19 of the formula IIb, chosen from:
6-methyl-4′-trifluoromethoxybiphenyl-2-carboxylic acid;
6-methoxy-4′-trifluoromethoxybiphenyl-2-carboxylic acid;
6-chloro-4′-trifluoromethoxybiphenyl-2-carboxylic acid;
4′-isobutyrylbiphenyl-2-carboxylic acid;
4′-(2-acetoxyethyl)biphenyl-2-carboxylic acid;
4′-(2-methoxycarbonylethyl)biphenyl-2-carboxylic acid;
4′-(2-ethylcarbamoyloxyethyl)biphenyl-2-carboxylic acid;
4′-(2-ethylcarbamoyloxyethyl-6-methylbiphenyl-2-carboxylic acid.

21- Compound of the formula IIId: 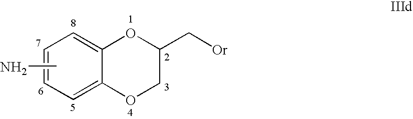
in which r represents (C₁-C₆)alkyl, preferably methyl, and NH₂ is located in position 6 or 7, with the exclusion of 2-ethoxymethyl-2,3-dihydro-benzo[1,4]dioxin-7-ylamine.

22- Compound according to claim 21 of the formula IIIc, chosen from:
3-methoxymethyl-2,3-dihydrobenzo[1,4]dioxin-6-ylamine; and
2-methoxymethyl-2,3-dihydrobenzo [1,4]dioxin-6-ylamine.

23- Compound of the formula XIa: 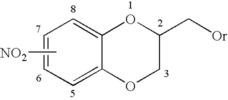
in which r represents (C₁-C₆)alkyl, preferably methyl, and NO₂ is located in position 6 or 7, with the exclusion of 2-ethoxymethyl-7-nitro-2,3-dihydro-benzo[1,4]dioxine.

24- Compound of the formula XIa according to claim 23, chosen from:
2-methoxymethyl-7-nitro-2,3-dihydrobenzo[1,4]dioxine;
2-methoxymethyl-6-nitro-2,3-dihydrobenzo [1,4]dioxine.

25- Compound of the formula IIIe: 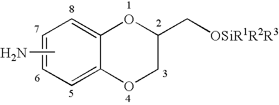
in which R¹, R² and R³ independently represent (C₁-C₆)alkyl and —NH₂ is located in position 6 or 7.

26- Compound of the formula IIIb according to claim 25, chosen from:
3-(tert-butyldimethylsilanyloxymethyl)-2,3-dihydrobenzo[1,4]dioxin-6-ylamine, and
2-(tert-butyldimethylsilanyloxymethyl)-2,3-dihydrobenzo [1,4]dioxin-6-ylamine.

27- Compound of the formula IVa: 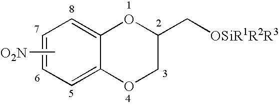
in which R¹, R² and R³ independently represent (C₁-C₆)alkyl; and NO₂ is located in position 6 or 7.

28- Compound of the formula IVa according to claim 27, chosen from:
tert-butyldimethyl(7-nitro-2,3-dihydrobenzo[1,4]dioxin-2-ylmethoxy)silane;
tert-butyldimethyl(6-nitro-2,3-dihydrobenzo[1,4]dioxin-2-ylmethoxy)silane.

29- Pharmaceutical composition comprising one or more compounds of the formula I as defined in claim 1, in combination with one or more excipients.

30- Use of a compound of the formula I according to claim 1, for the preparation of a pharmaceutical composition for inhibiting microsomal triglyceride transfer protein (MTP).

31- Use according to claim 29, characterised in that the said pharmaceutical composition is intended for the treatment of hypercholesterolaemia, hypertriglyceridaemia, hyperlipidaemia, pancreatitis, hyperglycaemia, obesity, atherosclerosis and diabetes-related dyslipidaemia.
